(19) United States
(12) Patent Application Publication
(10) Pub. No.: US 2011/0294874 A1
(43) Pub. Date: Dec. 1, 2011

(54) DIAGNOSIS AND THERAPY OF ORGAN DYSFUNCTION USING SPHINGANINE-1-PHOSPHATE

(75) Inventors: H. Thomas Lee, New York, NY (US); Mihwa Kim, Merrick, NY (US)

(73) Assignee: The Trustees of Columbia University in the City of New York, New York, NY (US)

(21) Appl. No.: 13/133,479

(22) PCT Filed: Dec. 9, 2009
(86) PCT No.: PCT/US09/67336

§ 371 Date: Aug. 17, 2011

Related U.S. Application Data
(60) Provisional application No. 61/201,354, filed on Dec. 9, 2008.

(51) Int. Cl. A61K 31/133; A61K 31/661; A61K 48/00; C12Q 1/02; G01N 30/02; C12Q 1/42; A61P 1/16; A61P 13/12; A61P 9/10; A61K 31/137; C12N 5/00

(52) U.S. Cl. 514/44 R; 514/653; 514/669; 514/114; 435/29; 435/375; 435/21; 73/61.52

(57) ABSTRACT

The invention relates to the treatment and diagnosis of organ dysfunction caused by ischemia reperfusion injury. In particular, the invention relates to sphinganine-1-phosphate, a sphingolipid metabolite, and its use in the diagnosing, preventing, and/or treating ischemia reperfusion-associated disorders, including, without limitation, disorders of the kidney, liver, lung, brain, and heart.

### FIELD OF THE INVENTION

The invention relates to the treatment and diagnosis of organ dysfunction caused by ischemia reperfusion injury. In particular, the invention relates to sphinganine-1-phosphate, a sphingolipid metabolite, and its use in the diagnosing, preventing, and/or treating ischemia reperfusion-associated disorders, including, without limitation, disorders of the kidney, liver, lung, brain, and heart.

### BACKGROUND OF THE INVENTION

Liver failure is the 12th leading cause of death and is often the result of liver transplantation complications including surgical complications (bleeding, vascular or biliary), primary non-function or poor early graft function infection, acute rejection, liver dysfunction due to ischemia reperfusion injury as well as consequences of liver ischemia reperfusion to other organs such as the kidney and lungs. Hepatic ischemia reperfusion injury is a major cause of acute liver failure and frequently follows hepatic resection, liver transplantation or portal vein reconstruction.

Current markers of liver injury include aspartate aminotransferase (AST), alanine aminotransferase (ALT), total Bilirubin (Tbili), alkaline phosphatase (ALP), INR and fibrinogen. ALT, also called serum glutamic pyruvate transaminase (SGPT) or alanine aminotransferase (ALAT), is an enzyme present in hepatocytes (liver cells). When a cell is damaged, it leaks this enzyme into the blood, where it is measured. The ALT level rises dramatically in acute liver damage, such as viral hepatitis or paracetamol (acetaminophen) overdose (usually above the normal range of 5-50 U/L). ALT as well as the other indicators measure the functional capacity of the liver but do not offer any therapeutic advantage.

Patients experiencing hepatic ischemia reperfusion injury also frequently developed acute renal failure or acute kidney injury (AKI). The incidence of renal failure in patients with acute liver failure ranges from 40-85% depending on the diagnostic criteria and etiology. Renal failure is a disease state in which renal functions are sufficiently damaged such that internal environment of the living body can no longer be maintained in normal conditions. In particular, acute renal failure involves a sudden loss of the kidneys' ability to excrete wastes, concentrate urine, and conserve electrolytes. Causes of acute renal failure include acute tubular necrosis (ATN), myoglobinuria (myoglobin in the urine), infections such as acute pyelonephritis or septicemia, urinary tract obstruction such as a narrowing of the urinary tract (stricture), tumor, kidney stones, nephrocalcinosis, enlarged prostate with subsequent acute bilateral obstructive uropathy, severe acute nephritic syndrome, disorders of the blood, malignant hypertension, and autoimmune disorders such as scleroderma. Other causes such as poisons and trauma, for example a direct and forceful blow to the kidneys, can also lead to renal failure.

Treatment for acute renal failure include administration of loop diuretics and osmotic diuretics, which are used in expectation of recovery of renal functions by increasing the flow in kidney tubules so as to wash away casts formed in the tubules and thereby prevent obstruction of the tubules. However, depending on the manner of use, these agents present the risk of inviting hearing disorders and the even more severe adverse side effects of heart failure and pulmonary edema.

Current markers of acute renal failure or acute kidney injury, including creatinine and cystatin C, are only detected much later after the onset of injury, and thus, do not offer any early prediction or detection. Creatinine is a break-down product of creatine phosphate in muscle, and is usually produced at a fairly constant rate by the body (depending on muscle mass). Creatinine is mainly filtered by the kidney, though a small amount is actively secreted. There is little-to-no tubular re-absorption of creatinine. If the filtering of the kidney is deficient, blood levels of creatinine rise. As a result, creatinine levels in blood (plasma) and urine may be used to calculate the creatinine clearance (CrCl), which reflects the glomerular filtration rate (GFR). The GFR is clinically important because it is a measurement of renal function. A more complete estimation of renal function can be made when interpreting the blood (plasma) concentration of creatinine along with that of urea. Measuring serum creatinine is a simple test and it is the most commonly used indicator of renal function. A rise in blood creatinine levels is observed only with marked damage to functioning nephrons. Therefore, this test is not suitable for detecting early stage kidney disease.

Renal failure after liver surgery often increases mortality and morbidity. Presently, no effective therapy currently exists to prevent this injury and the clinical management remains largely supportive and is limited to hydration, blood pressure support and hemodialysis.

The mechanism of renal injury following hepatic ischemia reperfusion is currently unknown. We developed a mouse model of AKI after hepatic ischemia reperfusion that shares similar histological and biochemical changes to that observed in human AKI associated with acute liver failure (Lee et al., 2009). Mice with AKI after hepatic ischemia reperfusion displayed marked renal vascular endothelial cell apoptosis and renal tubule F-actin disruption. Endothelial cell death due to apoptosis would impair defenses against leukocyte invasion into the kidney leading to further exacerbation of renal injury. This suggested that reducing endothelial cell death may limit renal injury associated with liver failure.

Ischemia reperfusion injury plays a role not only in liver failure and renal injury but it may also contribute to other organ dysfunctions. For example, ischemia reperfusion injury is often sustained in patients undergoing cardiopulmonary bypass, abdominal aortic occlusion and reperfusion, kidney ischemia and reperfusion during and after transplantation, partial nephrectomy and stroke.

Accordingly, there is a need for therapeutic and diagnostic agents for detecting and treating renal injury associated with liver failure as well as other disorders associated with ischemia reperfusion injury.

### SUMMARY OF THE INVENTION

In accordance with a first aspect of the present invention, there is provided a method for treating, inhibiting, or preventing renal and/or liver failure due to an ischemia reperfusion injury in a mammalian subject in need thereof, the method comprising the step of administering to the subject a therapeutically effective amount of a therapeutic agent selected from the group consisting of sphinganine-1-phosphate, sphinganine and a S1P1 receptor agonist. In certain embodiments of the invention, the S1P1 receptor agonist is selected from the group consisting of SEW2871 and FTY720. In certain embodiments of the invention, the ischemia reperfusion injury is associated with organ transplant, liver ischemia, kidney ischemia, stroke, abdominal aortic occlusion, abdominal aortic bleeding or perioperative ischemia due to cardiopulmonary bypass surgery. In certain embodiments of the invention, the organ transplant is selected from the group consisting of a liver transplant, a kidney transplant, a heart transplant, a lung transplant, a pancreas transplant, and an intestine transplant. In certain embodiments of the invention, the therapeutic agent is administered to the subject via parenteral administration. In certain embodiments of the invention, the parenteral administration is selected from the group consisting of intravenous administration, intraperitoneal administration, subcutaneous administration, intrarenal administration, intrahepatic administration and intramuscular administration. In certain embodiments, the therapeutic agent is administered prior to reperfusion. In certain embodiments of the invention, the mammalian subject is a human subject.

In accordance with a second aspect of the present invention, there is provided a method for treating, inhibiting, or preventing renal and/or liver failure due to an ischemia reperfusion injury in a mammalian subject in need thereof, the method comprising the step of increasing sphinganine-1-phosphate levels in the subject to a therapeutically effective level. In certain embodiments of the invention, the step of increasing sphinganine-1-phosphate levels comprises administering to the subject a sufficient dose of sphinganine-1-phosphate, sphinganine, sphingosine-1-phosphate, or sphingosine. In certain embodiments of the invention, the therapeutic agent is administered to the subject via parenteral administration. In certain embodiments of the invention, the parenteral administration is selected from the group consisting of intravenous administration, intraperitoneal administration, subcutaneous administration, intrarenal administration, intrahepatic administration and intramuscular administration. In certain embodiments of the invention, the therapeutic agent is administered prior to reperfusion. In certain embodiments of the invention, the step of increasing sphinganine-1-phosphate levels comprises administering to the subject a nucleic acid encoding a sphingosine kinase (SK), and allowing the sphingosine kinase protein to be expressed from the nucleic acid in an amount sufficient to increase sphinganine-1-phosphate levels in the subject to a therapeutically effective level. In certain embodiments, the SK is SK1 or SK2. In certain embodiments of the invention, the ischemia reperfusion injury is associated with organ transplant, liver ischemia, kidney ischemia, stroke, abdominal aortic occlusion, abdominal aortic bleeding or perioperative ischemia due to cardiopulmonary bypass surgery. In certain embodiments of the invention, the organ transplant is selected from the group consisting of a liver transplant, a kidney transplant, a heart transplant, a lung transplant, a pancreas transplant, and an intestine transplant. In certain embodiments of the invention, the nucleic acid encoding the sphingosine kinase is administered to the subject using a viral vector. In certain embodiments of the invention, the viral vector is selected from the group consisting of a retroviral vector, lentiviral vector, an adenoviral vector, a baculoviral vector, an Epstein Barr viral vector, a papovaviral vector, a vaccinia viral vector, and a herpes simplex viral vector. In certain embodiments of the invention, the viral vector is a lentiviral vector. In certain embodiments of the invention, the viral vector is administered to the subject via parenteral injection. In certain embodiments of the invention, the parenteral administration of the viral vector is intrarenal administration. In certain embodiments of the invention, the mammalian subject is a human subject.

In accordance with a third aspect of the present invention, there is provided a method of identifying a mammalian subject who is developing renal failure due to an ischemia reperfusion injury comprising the steps of determining the concentration of sphinganine-1-phosphate in a biological sample from said subject who has suffered an ischemia reperfusion injury; comparing said concentration of sphinganine-1-phosphate in said subject with a reference concentration of sphinganine-1-phosphate; wherein reduced sphinganine-1-phosphate concentration in said subject compared to said reference concentration is indicative that said subject is developing renal failure. In certain embodiments of the invention, the reference concentration is the concentration in a biological sample from at least one subject who has not suffered an ischemia reperfusion injury. In certain embodiments of the invention, the reference concentration is the concentration of sphinganine-1-phosphate in a biological sample from the subject prior to suffering from said ischemia reperfusion injury. In certain embodiments of the invention, the ischemia reperfusion injury is associated with organ transplant, liver ischemia, kidney ischemia, stroke, abdominal aortic occlusion, abdominal aortic bleeding or perioperative ischemia due to cardiopulmonary bypass surgery. In certain embodiments of the invention, the organ transplant is selected from the group consisting of a liver transplant, a kidney transplant, a heart transplant, a lung transplant, a pancreas transplant, and an intestine transplant. In certain embodiments of the invention, the mammalian subject is a human subject. In certain embodiments, the biological sample is selected from the group consisting of blood, urine, saliva, tissue sample from a liver, tissue sample from a kidney, tissue sample from a heart, tissue sample from a muscle and tissue sample from a blood vessel.

In accordance with an fourth aspect of the present invention, there is provided a method for reducing, inhibiting or preventing kidney endothelial cell injury, the method comprising administering an effective amount of sphinganine-1-phosphate to the endothelial cells. In certain embodiments, the endothelial cell injury is associated with apoptosis, necrosis or inflammation. In certain embodiments of the invention, the kidney endothelial cells are mammalian kidney endothelial cells. In certain embodiments of the invention, the mammalian kidney endothelial cells are human kidney endothelial cells.

### BRIEF DESCRIPTION OF THE DRAWINGS

FIG. 1 is a graph showing the correlation between the severity of liver dysfunction (as measured by serum alanine aminotransferase (ALT)) and the degree of acute kidney injury (AKI) (as measured by serum creatinine (Cr)) 24 hours following liver ischemia and reperfusion (p<0.0001 and r²=0.8925).

FIG. 2 is a graph showing plasma IL-6 and TNF-α levels in mice subjected to liver IR with and without sphinganine 1-phosphate treatment.

FIG. 3 shows representative photomicrographs of kidneys from mice subjected to sham-operation or to liver IR. Sham operated animals showed normal-appearing glomeruli (A) and tubules (B). Following liver IR, many glomeruli display prominent hyperplasia of the juxtaglomerular apparatus (arrow) located at the glomerular hilus (C). There is focal coarse clear cytoplasmic vacuolization (arrows) of proximal tubular epithelial cells (D). Some cortical tubular cells display acute epithelial injury with cellular condensation and cytoplasmic hypereosinophilia (arrows) (E). There is focal apoptosis of interstitial capillary endothelial cells (circle) with marked vascular stasis within the interstitial capillary lumina (arrows) (F).

FIG. 4 shows renal endothelial cell apoptosis after liver IR. C57BL/6 mice were subjected to 60 minutes liver ischemia and 24 hours reperfusion and serial kidney sections from the mice were stained with TUNEL (A) or with CD34 (an endothelial cell marker, B). Identical cells are labeled a-k in the TUNEL and immunohistochemical images of the same microscopic field. Representative of 4 serial sections stained.

FIG. 5 shows a representative high performance liquid chromatography (HPLC) analysis for S1P and Sg1P levels in plasma of mice subjected to liver ischemia or to sham operation, with C17-sphingosine 1-phosphate used as an internal standard (Int Std), with which the amount of S1P as well as Sg1P present in the original sample was quantified.

FIG. 6 shows the effect of Sg1P administration on hepatic and renal injury after 60 minutes of ischemia followed by 24 hours reperfusion. Administration of Sg1P (0.1 mg/kg Sg1P intravenous 15 min. before and 2 hr after liver reperfusion) in liver IR mice resulted in lower plasma ALT levels compared to matching controls treated with vehicle. Administration of Sg1P in liver IR mice also reduced the rise in plasma creatinine levels observed in liver IR mice compared to matching controls treated with vehicle.

FIG. 7 shows photomicrographs (400×) of kidney H&E staining from C57BL/6 mice after liver IR, with and without Sg1P treatment.

FIG. 8 shows renal injury scores (scale 0-3) following liver IR with and without Sg1P treatment, based on H&E stained kidney samples (as exemplified in FIG. 7).

FIG. 9 shows caspase-3 fragmentation in liver (A) and kidney (B) following liver IR, with and without Sg1P treatment.

FIG. 10 shows fluorescent photomicrographs (400×) of phalloidin staining to visualize F-actin in liver and kidney tissue samples from mice subjected to liver IR, with and without Sg1P treatment.

FIG. 11 shows Evans Blue Dye (EBD) extravasation measured in liver and kidney tissues in mice subjected to liver IR with and without Sg1P treatment,

FIG. 12 shows the expression level of pro-inflammatory proteins in liver (A) and kidney (B) following liver IR, based on densitometric quantification of relative mRNA band intensities normalized to GAPDH.

FIG. 13 shows the dose response range of Sg1P during liver (hepatic) ischemia/reperfusion. The sham-operation group and vehicle-treated hepatic IR group were merged in untreated group (0 mg/kg, N=22). The following dosages of Sg1P were tested: 0.01 mg/kg intravenous (i.v.) prior to reperfusion followed by 0.02 mg/kg subcutaneous (s.c.) 2 hours after reperfusion (N=8); 0.05 mg/kg i.v. prior to reperfusion followed by 0.1 mg/kg s.c. 2 hours after reperfusion (N=7); 0.1 mg/kg i.v. prior to reperfusion followed by 0.2 mg/kg s.c. 2 hours after reperfusion (N=23); 0.5 mg/kg i.v. prior to reperfusion followed by 1.0 mg/kg s.c. 2 hours after reperfusion (N=4); and 1.0 mg/kg i.v. prior to reperfusion followed by 2.0 mg/kg s.c. 2 hours after reperfusion (N=3). The effect of the different Sg1P dosages on hepatic and renal injury were measured by serum ALT and Cr levels, respectively. The dosage of 0.1 mg/kg i.v. prior to reperfusion followed by 0.2 mg/kg s.c. 2 hours after reperfusion was found to be the most effective dose for treating both kidney and liver failure.

FIG. 14 shows an increase in survival rate in mice treated with Sg1P after liver IR. The survival rates of Sg1P-treated mice 24 hours post liver IR was ˜90% (N=10), compared to ˜70% in the control C57BL/6 mice post liver IR (N=10). After 1 week, approximately 20% of mice subjected to liver IR remained alive, compared to a survival rate of approximately 50% in mice subjected to liver IR that received Sg1P.

FIG. 15 shows the level of liver injury (ALT) and kidney injury (Creatinine) in mice subjected to liver IR, when treated with Sg1P before or after reperfusion.

FIG. 16 shows the change in effectiveness of Sg1P treatment in suppressing liver IR-induced kidney (A) and liver (B) injury in mice when pre-treated with W146 (S1P1 antagonist), JTE-013 (S1P2 antagonist), or BML-241 (S1P3) antagonist.

FIG. 17 shows the change in effectiveness of Sg1P treatment in suppressing liver IR-induced kidney (A) and liver (B) injury in mice when pretreated with pertussis toxin (an inhibitor of Gi/o signaling), PD98059 (a selective MEK1 inhibitor), wortmannin (a selective PI3K inhibitor) or L-NIO (a selective eNOS inhibitor) prior to sphinganine 1-phosphate treatment.

FIG. 18 shows, as assessed by the scoring of H&E stained mouse kidney sections, the change in effectiveness of Sg1P treatment in suppressing liver IR-induced kidney damage when combined with the blockade of S1P1 receptors, MEK1, PI3K or Gi/o by pre-treating mice with W146, PD98059, wortmannin or pertussis toxin, respectively.

FIG. 19 shows Sg1P-induced phosphorylation of ERK/MAPK, Akt and phosphorylation of HSP27 in the liver and kidney of mice.

FIG. 20 shows Sg1P-induced expression of HSP27 in the liver and kidney of mice.

FIG. 21A shows the effect of Sg1P on the TNF-α-induced albumin leak (at 50 ng/mL and 100 ng/mL) in immortalized human umbilical vein endothelial cells (IHUVEC) monolayers.

FIG. 21B shows the effect of Sg1P on the thrombin-induced albumin leak (at 5 U/mL) in immortalized human umbilical vein endothelial cells (IHUVEC) monolayers.

FIG. 22 shows that treatment with Sg1P (1 μM) induced the phosphorylation of anti-apoptotic kinases ERK and Akt, as well as increased total and phosphorylated HSP27 in human renal endothelial cells (HRGEC).

FIG. 23 shows that treatment with Sg1P (1 μM) induces expression of HSP27 in human renal endothelial cells (HRGEC).

FIG. 24 shows fluorescent phalloidin staining in HRGEC cells treated with TNF-α, with and without Sg1P.

FIG. 25 shows western blots HUVEC (human unbilical vein endothelial cells), showing the effect of SK overexpression on Caspase 3 and PARP cleavage.

FIG. 26 shows the blood levels of Sg1P levels in human liver transplant recipients before and after the transplantation surgery. Serum Sg1P average levels dropped in patients following liver transplantation and remained at a low level until at least 48 hours post transplantation whereas serum S1P average levels remained the same.

FIG. 27 shows that blood Sg1P levels are stable in patients that received a live-donor liver (LR, N=5), whereas in patients receiving cadaveric liver (Cadaveric, N=7), Sg1P levels decrease, and that S1P levels measured from the same samples stayed stable throughout the pre- and post-operation periods.

FIG. 28 shows that blood Sg1P levels are decreased in patients who underwent coronary bypass surgery, and that Sg1P levels are correlated with post-operative outcome. S1P levels measured from the same samples remained relatively the same throughout the pre- and post-operation periods.

### DETAILED DESCRIPTION OF THE INVENTION

### Overview

Liver failure is a frequent cause of renal failure. In fact, a large percentage of patients with end stage liver disease also develop renal failure. Moreover, renal failure is a frequent and devastating complication after liver surgery including orthotopic liver transplantation, hepatic resection or prolonged portal vein occlusion. We recently developed a novel mouse model of kidney failure after liver injury to better study this major clinical problem. Based on the data generated from the mouse model, we discovered that plasma levels of a novel endogenous sphingolipid metabolite, spinganine-1-phosphate (Sg1P), were significantly lower in mice after liver ischemia reperfusion. We also discovered that exogenous administration of Sg1P before liver ischemia reperfusion rescues renal as well as liver function in mice. We further discovered that significantly lower Sg1P levels were also detected in blood samples from human patients subjected to liver transplantation and that the Sg1P levels correlated with patient outcome.

Thus, the present invention relates to the novel finding that administration of a therapeutically effective amount of Sg1P is useful for treating, inhibiting or preventing renal and liver failure due to an ischemia reperfusion injury.

We also discovered that mice genetically deficient in sphingosine kinase 1 or 2 (SK1 or SK2), the enzymes that generate Sg1P from sphinganine, showed increased renal injury and liver injury after liver ischemia reperfusion. Conversely, we found that overexpressing SK1 or SK2 in the kidney reduced renal injury and liver injury after liver ischemia reperfusion. Thus, the present invention also relates to the novel finding that administration of a nucleic acid encoding SK and allowing the SK protein to be expressed from the nucleic acid in an amount sufficient to increase Sg1P levels to a therapeutically effective level is useful for treating, inhibiting or preventing renal and/or liver failure due to an ischemia reperfusion injury.

The present invention also relates to the novel finding that increasing Sg1P levels in a subject is useful for treating, inhibiting or preventing renal and/or liver failure due to an ischemia reperfusion injury. Increasing Sg1P levels in a subject can be achieved in many ways, including, but not limited to: administration of sphinganine-1-phosphate; administration of sphinganine; administration of sphingosine-1-phosphate; administration of sphingosine; and administration of a nucleic acid encoding SK and allowing the SK protein to be expressed from the nucleic acid.

The present invention also relates to the novel finding that administration of a S1P1 receptor agonist is useful for treating, inhibiting or preventing renal and/or liver failure due to an ischemia reperfusion injury.

Also provided are novel methods of identifying a subject who is developing renal failure due to an ischemia reperfusion injury.

We also discovered that Sg1P reduced renal vascular endothelial cell injury, which may limit renal injury associated with liver failure. Accordingly, novel methods for reducing or preventing kidney endothelial cell injury are also described.

It will be understood by one of ordinary skill in the art that the compositions and methods described herein may be adapted and modified as is appropriate for the application being addressed and that the compositions and methods described herein may be employed in other suitable applications, and that such other additions and modifications will not depart from the scope hereof.

Throughout this application, various documents are referenced. Full citations for these documents are presented immediately before the listing of embodiments. Disclosures of these documents in their entireties are hereby incorporated by reference into this application.

### DEFINITIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990). The nomenclatures used in connection with, and the laboratory procedures and techniques of, molecular and cellular biology and medicinal and pharmaceutical chemistry described herein are those well known and commonly used in the art. Standard techniques are used for chemical syntheses, chemical analyses, pharmaceutical preparation, formulation, and delivery, and treatment of patients.

Throughout this specification, the word “comprise” or variations such as “comprises” or “comprising” will be understood to imply the inclusion of a stated integer or group of integers but not the exclusion of any other integer or group of integers.

The singular forms “a,” “an,” and “the” include plural reference unless the context clearly dictates otherwise.

The term “including” is used to mean “including but not limited to”. “Including” and “including but not limited to” are used interchangeably.

The term “agent” is used herein to denote a chemical compound (such as an organic or inorganic compound, a mixture of chemical compounds, a biological macromolecule (such as a nucleic acid, an antibody, a protein or portion thereof, e.g., a peptide, a lipid, a carbohydrate), or an extract made from biological materials such as bacteria, plants, fungi, or animal (particularly mammalian) cells or tissues. Agents include, for example, agents which are known with respect to structure and/or function, and those which are not known with respect to structure or function. The activity of such agents may render it suitable as a “therapeutic agent” which is a biologically, physiologically, or pharmacologically active substance (or substances) that acts locally or systemically in a subject. Agents can comprise, for example, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes). Agents may also comprise alcohols, alkyl halides, amines, amides, esters, aldehydes, ethers and other classes of organic agents.

A “patient”, “subject”, or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

The term “mammalian subject” shall include, but is not limited to, humans, non-human primates, mouse, cow, pig, goat, cat, rabbit, rat, guinea pig, hamster, horse, monkey, sheep, or other non-human mammals.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semi-synthetic, or synthetic origin which either does not occur in nature or is linked to another polynucleotide in a non-natural arrangement.

The term “prophylactic” or “therapeutic” treatment is art-recognized and refers to administration of a drug to a patient. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutically effective amount” is used herein to refer to an amount of an agent that is effective, upon single or multiple dose administration to a subject (such as a human patient) at treating, inhibiting, preventing, curing, delaying, alleviating, reducing the severity of, and/or ameliorating at least one symptom of a disorder or recurring disorder, or prolonging the survival of the subject beyond that expected in the absence of such treatment. The precise effective amount needed for a subject will depend upon the subject's size and health, the nature and extent of the condition, and the therapeutics or combination of therapeutics selected for administration. The effective amount for a given situation can be determined by routine experimentation.

The term “treatment” refers to a therapeutic or preventative measure. The treatment may be administered to a subject having a medical disorder or who ultimately may acquire the disorder, in order to prevent, cure, delay, alleviate, reduce the severity of, and/or ameliorate one or more symptoms of a disorder or recurring disorder, or in order to prolong the survival of a subject beyond that expected in the absence of such treatment.

The terms “inhibiting or preventing” the onset of a disorder shall refer to either lessening the likelihood of the disorder's onset, or preventing the onset of the disorder entirely Inhibiting the onset of a disorder may also mean preventing its onset entirely.

The terms “reducing or treating or alleviating” a subject afflicted with a disorder shall mean causing the subject to experience a reduction, remission or regression of the disorder and/or its symptoms.

The term “administering” or “administration” is used herein to denote delivering in a manner which is effected or performed using any of the various methods and delivery systems known to those skilled in the art. Administering can be performed, for example, topically, orally, via implant, transdermally, or parenterally. Parenteral administration can be performed transmucosally, intravenously, intramuscularly, subcutaneously, intraperitoneally, orintrathecally. Administering can also be performed, for example, once, a plurality of times, and/or over one or more extended periods.

The term “renal failure”, “renal injury”, “kidney failure” or “kidney injury” refers to a situation in which the kidney(s) fails to function adequately, i.e., an acute or chronic loss of a kidney's ability to excrete wastes, concentrate urine and/or conserve electrolytes. Renal failure can be divided into two types: acute and chronic forms. Biochemically, it is typically detected by an elevated serum creatinine Renal failure may also be described as a decrease in the glomerular filtration rate. Renal failure results in abnormal fluid levels in the body, deranged acid levels, hematuria, and abnormal levels of potassium, calcium, and phosphate.

The term “ischemia reperfusion” injury shall refer to injury to tissue or organ sustained during ischemia due to lack of oxygen and ATP synthesis followed by injury sustained during reperfusion due to oxidative stress when blood supply returns to tissue or organ after a period of ischemia. During ischemia reperfusion injury, the absence of oxygen creates and nutrients from blood a condition in which the restoration of circulation (that is, reperfusion) results in inflammation and oxidative damage (oxidative stress) rather than restoration of normal function.

The term “liver failure”, “liver injury”, “hepatic failure” or “hepatic injury” shall refer to the inability of the liver to perform its normal synthetic and metabolic function as part of normal physiology.

The term “viral vector” shall refer to a nucleic acid of viral origin encoding a nucleic acid of interest and/or a protein of interest, which nucleic acid, when placed in a cell, permits the expression of the nucleic acid or protein of interest. Viral vectors are well known in the art.

I. Sphinganine-1-Phosphate and its Correlation with Renal and Liver Injury Following Liver Ischemia Reperfusion

As described in more detail in Example 1, we developed a murine model of acute kidney injury (AKI) after liver ischemia reperfusion (IR) that mimics both the histological and biochemical changes observed with human AKI associated with acute liver failure (Lee et al., 2009). As described in more detail in Example 2, a direct relationship between the severity of liver dysfunction (as determined by ALT levels) and the degree of AKI (as determined by Cr levels) was found in mice 24 hours after liver ischemia reperfusion (IR). Liver IR-induced AKI in mice also shared similar histological changes observed in human AKI associated with liver failure. For example, marked renal endothelial cell apoptosis and severe loss of renal F-actin proximal tubules were observed. We also observed changes in liver and kidney vascular permeability after liver IR in the mice, suggesting that endothelial cell damage and injury may be responsible for the renal injury associated with liver IR.

Sphingolipids are ubiquitous and essential structural, as well as functional, components of the plasma membrane. In addition, sphingolipid metabolites have important biological roles in various physiological as well as pathophysiological events, such as regulation of immune system including the growth and invasiveness of human cancer cells. Sphingolipid metabolites, such as ceramide and sphingosine-1-phosphate, have been shown to be important mediators in the signaling cascades involved in apoptosis, proliferation, and stress responses.

Sphingolipids are any lipid containing the long-chain amino alcohol sphingosine or a variation of it, such as dihydrosphingosine, phytosphingosine or dehydrophytosphingosine. Sphingosine itself is synthesized by condensing a long-chain fatty acid with the amino acid serine.

Sphingosine is converted into a variety of derivatives to form the family of sphingolipids. The simplest form is a ceramide which contains a sphingosine and a fatty acid residue joined by an amide linkage. Ceramide is the basic building block of practically all of the naturally occurring sphingolipids. It can be further modified by the addition of a phosphorylcholine at the primary alcohol group to form sphingomyelin, a ubiquitous phospholipid in the plasma membranes of virtually all cells. Modification of a ceramide by addition of one or more sugars at the primary alcohol group converts it to a glycosphingolipid. In addition, Palmitoyl-CoA and I serine can by synthesized into 3-ketosphinganine, the enzymatically reduced by 3-ketosphinganine reductase into sphinganine Sphinganine can be joined by a fatty acid residue by an amide linkage to form dihyrdroceramide (catalyzed by ceramide synthase), which can then be converted into ceramide through dihydroceramide desaturase. Sphingosine and sphinganine can both be phosphorylated by sphingosine/sphinganine kinase to form sphingosine-1-phosphate (S1P) or sphinganine-1-phosphate (Sg1P), respectively. They can be dephosphorylated by S1P phosphatase. Although sphinganine-1-phosphate (Sg1P) is structurally similar to sphingosine-1-phosphate (S1P), Sg1P differs from S1P by being cell impermeable and lacking the trans double bond at the 4 position.

Sphingosine kinase (SK) is a lipid kinase with two mammalian isoforms (SK1 and SK2), which catalyzes the ATP dependent phosphorylation of sphingosine and sphinganine to form sphingosine-1-phosphate (S1P) and sphinganine-1-phosphate (Sg1P), respectively. It is also known as sphinganine kinase.

S1P has emerged as an important mediator of a variety of biological processes, including cell growth and survival, as well as inflammation. Because S1P has been extensively studied as an endogenous molecule playing a protective role against endothelial dysfunction, we measured the level of S1P to determine if a depletion of S1P was responsible for the increased renal endothelial damage after liver IR.

Sphingolipids may be measured using methods including, but not limited to, radiolabeling assays (involving radiolabeled substrates or radioreceptor binding assays), enzymatic assays (by enzymatic dephosphorylation of the substrate followed by rephosphorylation in the presence of [γ-³²P]ATP) and chromatography-based assays (including high-performance liquid chromatography [HPLC]). The use of HPLC for quantitative analysis of sphingolipids is preferred as the method is able to resolve structurally similar compounds, e.g., sphingosine-1-phosphate (S1P) and sphinganine-1-phosphate (Sg1P). HPLC detection of S1P was performed as described by Min et al. (2002) using two steps of sample pretreatment: enzymatic dephosphorylation of S1P by alkaline phosphatase and subsequent analysis of o-phthalaldehyde derivatives of the liberated sphingosine bases by HPLC. By introducing C17 S1P as an internal standard, S1P present in a sample can be quantified on a C18 reversed-phase column with a simple mobile phase of acetonitrile:deionized distilled water (90:10, v/v).

As described in more detail in Example 3, plasma levels of S1P did not change in mice after liver ischemia reperfusion. However, plasma levels of Sg1P decreased significantly in mice subjected to liver IR. Not only were plasma levels of Sg1P decreased following liver IR but renal levels of Sg1P were also reduced after liver IR, although S1P levels remained the same. Therefore, after liver IR, there was a selective reduction in Sg1P levels.

Similar to the mouse model, we also observed that serum Sg1P levels, and not S1P levels, dropped in human patients following liver transplantation and remained at a low level until at least 48 hours post transplantation (as described in more detail in Example 11). We also observed that a bigger post-operative drop in serum Sg1P correlated with worse outcome. Thus, Sg1P levels may be used as a diagnostic for monitoring the renal health of liver transplantation patients as well as a predictor of transplantation success and patient outcome/mortality.

### II. Therapeutic Uses of Sphinganine-1-Phosphate

We also discovered that exogenous Sg1P provides protection from renal and hepatic injury after liver IR. As described in more detail in Example 4, mice injected with exogenous Sg1P not only displayed significantly improved renal function but also showed reduced liver dysfunction. Furthermore, renal histology revealed that the vascular endothelial cell apoptosis that was prominent after liver IR in vehicle-treated animals, was significantly reduced in mice treated with Sg1P and subjected to liver IR. Moreover, endothelial dysfunction was significantly reduced in the kidneys as well as livers of mice treated with Sg1P. Therefore, liver IR significantly and selectively depletes plasma Sg1P, and exogenous Sg1P provides protection from renal as well as hepatic injury after liver IR. In addition, the administration of sphinganine, S1P, or sphingosine, each of which increases serum Sg1P levels, was also found to provide protection from renal and hepatic injury in mice after liver IR.

It may also be desirable in the treatment of renal or liver failure to modulate a particular biological pathway that is critical for disease progression, by modulating functions of ligands and their receptors, modulating receptor activity and the activity of down stream signaling proteins, and/or modulating redundant elements of a pathway. Such methods may be used for treating renal or liver failure including those that involve the sphinganine-1-phosphate pathway.

We demonstrated that modulation of the receptor activity may be useful for treatment of renal or liver failure due to ischemia reperfusion injury. Sphingolipid receptors represent a family of G protein-coupled receptors that mediate a broad range of cellular effects, including growth and migration. There are five sphingosine-1-phosphate (S1P) receptors (S1P1-S1P5).

As described in more detail in Example 5, the therapeutic action of Sg1P administration in reducing renal and hepatic failure due to liver IR was mediated by the specific activation of the S1P1 receptor by Sg1P. We found that, in contrast to the reduced renal and hepatic injury observed following Sg1P administration alone, administration of Sg1P combined with an S1P1/3 antagonist (VPC 23019) failed to reduce renal injury induced by liver IR. Similar results were obtained when Sg1P treatment was combined with the administration of W146, a more specific S1P1 antagonist. Further, treatment with S1P1 receptor agonist mimicked the effect of Sg1P in treating renal and liver failure due to Liver IR. In contrast, the administration of specific S1P2 or S1P3 receptor blockers did not block the therapeutic action of Sg1P. These results demonstrate that the therapeutic activity of Sg1P is mediated through specific activation of the S1P1 receptor, and those methods which modulate the sphinganine-1-phosphate pathway, such as activating S1P1 receptor activity, is useful for treating renal or liver failure due to ischemia reperfusion injury.

Based on our novel discoveries, the present invention relates to the use of sphinganine-1-phosphate in treating, inhibiting or preventing renal and/or hepatic failure due to an ischemia reperfusion injury.

In one embodiment of the invention, the invention relates to a method for reducing, treating or alleviating renal and/or hepatic failure due to an ischemia reperfusion injury in a mammalian subject in need thereof, the method comprising the step of administering to the subject a therapeutically effective amount of sphinganine-1-phosphate.

The present invention also relates to a method for treating, inhibiting, or preventing renal and/or hepatic failure due to a liver ischemia or reperfusion injury in a mammalian subject in need thereof, the method comprising the step of administering to the subject a therapeutically effective amount of a therapeutic agent selected from the group consisting of sphinganine-1-phosphate, sphinganine, sphingosine-1-phosphate, sphingosine and a S1P1 receptor agonist. In certain embodiments of the invention, the S1P1 receptor is selected from the group consisting of SEW2871 and FTY720.

In certain embodiments of the invention, the ischemia reperfusion injury that causes renal failure or liver failure includes, without limitation, ischemia reperfusion injury that is associated with organ transplant, liver ischemia, kidney ischemia, stroke, abdominal aortic occlusion, abdominal aortic bleeding or perioperative ischemia due to cardiopulmonary bypass surgery. In certain embodiments of the invention, the organ transplant may include, without limitation, transplants of the liver, kidney, heart, lung, brain, pancreas and intestine. In preferred embodiments, the invention relates to organ transplants of the liver, kidney, heart, lung, pancreas and intestine.

The methods described herein for treating a subject suffering from renal failure due to an ischemia reperfusion injury may be used for the prophylactic treatment of individuals who have been diagnosed or predicted to be at risk for developing renal failure due to an ischemia reperfusion injury. The methods described herein for treating a subject suffering from liver failure due to a liver ischemia or reperfusion injury may also be used for the prophylactic treatment of individuals who have been diagnosed or predicted to be at risk for developing liver failure due to a liver ischemia or reperfusion injury.

Thus, in one embodiment of the invention, a composition comprising at least one therapeutic agent selected from the group consisting of sphinganine-1-phosphate, sphinganine, sphingosine-1-phosphate, sphingosine, and a S1P1 receptor agonist is administered in an amount and dose that is sufficient to treat, inhibit, delay, slow, or prevent the onset of renal or liver failure due to an ischemia reperfusion injury, or related symptoms, or to reverse renal or liver failure due to an ischemia reperfusion injury. It is understood that an effective amount of a composition for treating a subject who has been diagnosed with, or predicted to be at risk for developing, renal or liver failure due to an ischemia reperfusion injury is a dose or amount that is in sufficient quantities to treat a subject or to treat the disorder itself.

In certain embodiments of the invention, the therapeutic agent may be formulated with a pharmaceutically acceptable carrier. For example, the therapeutic agent can be administered alone or as a component of a pharmaceutical formulation (therapeutic composition). The therapeutic agent may be formulated for administration in any convenient way for use in human medicine.

In certain embodiments of the invention, the therapeutic methods of the invention include administering the composition topically, systemically, or locally. For example, therapeutic compositions of the invention may be formulated for administration by, for example, injection (e.g., intravenously, subcutaneously, or intramuscularly), inhalation or insufflation (either through the mouth or the nose) or oral, buccal, sublingual, transdermal, nasal, or parenteral administration. The compositions described herein may be formulated as part of an implant or device. When administered, the therapeutic composition for use in this invention is in a pyrogen-free, physiologically acceptable form. Further, the composition may be encapsulated or injected in a viscous form for delivery to the site where the target cells are present. Techniques and formulations generally may be found in Remington's Pharmaceutical Sciences, Meade Publishing Co., Easton, Pa. In addition to the therapeutic agent, additional therapeutically useful agents may optionally be included in any of the compositions described herein. Furthermore, therapeutically useful agents may, alternatively or additionally, be administered simultaneously or sequentially with the therapeutic agent according to the methods of the invention.

In certain embodiments of the invention, compositions comprising the therapeutic agent can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more compositions comprising the therapeutic agent may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In certain embodiments, pharmaceutical compositions suitable for parenteral administration may comprise the therapeutic agent in combination with one or more pharmaceutically acceptable sterile isotonic aqueous or non-aqueous solutions, dispersions, suspensions or emulsions, or sterile powders which may be reconstituted into sterile inject able solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

A composition comprising the therapeutic agent may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person of ordinary skill in the art, such as a physician, is able to determine the required amount to treat the subject. It is understood that the dosage regimen will be determined for an individual, taking into consideration, for example, various factors that modify the action of the therapeutic agent, the severity or stage of the disease, route of administration, and characteristics unique to the individual, such as age, weight, and size. In certain embodiments, the dosage can range from about 0.001 mg/kg to about 1 mg/kg body weight of the subject, or from about 0.001 mg/kg to about 0.01 mg/kg, from about 0.01 mg/kg to about 0.025 mg/kg, from about 0.025 mg/kg to about 0.05 mg/kg, from about 0.05 mg/kg to about 0.1 mg/kg, from about 0.1 mg/kg to about 0.25 mg/kg, from about 0.25 mg/kg to about 1.0 mg/kg or greater. In a certain embodiment, a composition comprising the therapeutic agent is administered in a range from about 0.01 mg/kg to 0.1 mg/kg.

The dose can be delivered continuously, or at periodic intervals (e.g., on one or more separate occasions). Desired time intervals of multiple doses of a particular composition can be determined by routine experimentation by one skilled in the art. For example, the compound may be delivered hourly, daily, weekly, monthly, yearly (e.g. in a time release form) or as a one time delivery. If administered orally or topically, such a preparation can be administered 1 to 6 times per day for a period of 1-4 weeks, 1-3 months, 3-6 months, 6-12 months, 1-2 years, or more, up to the lifetime of the patient. If administered by injection, the composition comprising the therapeutic agent can be delivered one or more times periodically throughout the life of a patient. For example, the composition comprising the therapeutic agent can be delivered once per year, once every 6-12 months, once every 3-6 months, once every 1-3 months, once every 1-4 weeks, one or more times per day. Alternatively, more frequent administration may be desired. If administered by an implant or device, the composition comprising the therapeutic agent can be administered one time, or one or more times periodically throughout the lifetime of the patient as necessary.

In certain aspects, the present invention provides gene therapy for increasing the in vivo production of sphinganine-1-phosphate. One means of accomplishing this is by increasing the in vivo production and activity of sphingosine kinase (SK), which is the enzyme that synthesizes sphinganine-1-phosphate. There are two isoforms of SK (SK1 and SK2) and we have demonstrated that mice genetically deficient for either SK1 or SK2 displayed a further reduction in plasma levels of sphinganine-1-phosphate, which correlated with renal and liver injury (see Example 6). We also demonstrated that administration of sphinganine-1-phosphate to these SK1- or SK2-deficient mice were able to increase sphinganine-1-phosphate levels and reduce renal as well as liver injury (see Examples 6 and 7).

In one embodiment, the invention provides a method for treating, inhibiting, or preventing renal failure due to an ischemia reperfusion injury in a mammalian subject in need thereof, the method comprising the step of increasing sphinganine-1-phosphate levels in the subject to a therapeutically effective level. In certain embodiments of the invention, the step of increasing sphinganine-1-phosphate levels comprises administering to the subject a sufficient dose of sphinganine-1-phosphate, sphinganine, S1P, or sphingosine. In certain embodiments of the invention, the step of increasing sphinganine-1-phosphate levels comprises administering to the subject a nucleic acid encoding a sphingosine kinase, and allowing the sphingosine kinase protein to be expressed from the nucleic acid in an amount sufficient to increase sphinganine-1-phosphate levels in the subject to a therapeutically effective level. Such gene-based therapies achieve its therapeutic effect by introduction of a polynucleotide or nucleic acid sequence that encodes a sphingosine kinase into cells or tissues. Delivery of sphingosine kinase polynucleotide or nucleic acid sequences can be achieved using a recombinant expression vector such as a chimeric virus.

Various viral vectors which can be utilized for gene therapy as taught herein include adenovirus, herpes virus, vaccinia, or an RNA virus such as a retrovirus. A retroviral vector may be a derivative of a murine or avian retrovirus. Examples of retroviral vectors in which at least a single foreign gene can be inserted include, but are not limited to: Moloney murine leukemia virus (MoMuLV), Harvey murine sarcoma virus (HaMuSV), murine mammary tumor virus (MuMTV), Rous Sarcoma Virus (RSV) and lentivirus. A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide or nucleic acid sequences can be inserted into the retroviral genome or attached to a viral envelope to allow target specific delivery of the retroviral vector containing the polynucleotide or nucleic acid that encodes sphingosine kinase.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

In certain embodiment of the present invention, the viral vectors include, but are not limited to, an adenoviral vector, an adeno-associated viral vector, a baculoviral vector, an Epstein Barr viral vector, papovaviral vector, a vaccinia viral vector, a retroviral vector, a lentiviral vector, a herpes viral vector, polyoma viral vector or hepatitis B viral vector. In preferred embodiments, the viral vector is a lentiviral vector.

The methods described herein may also comprise the administration of a one or more therapeutic agents selected from the group consisting of sphinganine-1-phosphate, sphinganine, sphingosine-1-phosphate, or sphingosine in combination. The methods described herein may also comprise the administration of the therapeutic agent in combination with one or more additional therapeutic agents. The term “in combination”, in this context, means that the therapeutic agent and the additional therapeutic agent are given as part of a treatment regimen. In some embodiments, the therapeutic agents are administered substantially contemporaneously, either simultaneously or sequentially, including one being a pretreatment in relation to the other. In some embodiments, in which administration is sequential, at the onset of administration of the second agent, the first of the two agents is still detectable at effective concentrations at the site of treatment. In another embodiment, if given sequentially, at the onset of administration of the second compound, the first of the two compounds is not detectable at effective concentrations at the site of treatment. In certain embodiments, the methods of the invention comprise administering to a subject a therapeutically effective amount of a therapeutic agent in combination with one or more additional agents that modulate endothelial cell function. In certain embodiments, the additional agent may be an anti-apoptotic or an anti-inflammatory agent. In certain embodiments, the additional agent may be activated protein C.

The methods described herein may also be useful for treating and diagnosing other organ dysfunctions caused by ischemia reperfusion injury. In particular, the present invention relates to sphinganine-1-phosphate and its use in diagnosing, preventing, and/or treating ischemia reperfusion-associated injury, including, without limitation, kidney injury (kidney ischemia), liver injury (liver ischemia), lung injury (lung ischemia), heart injury, brain injury (stroke), abdominal aortic occlusion, abdominal aortic bleeding and perioperative ischemia due to cardiopulmonary bypass surgery. For example, patients undergoing cardiac bypass surgery sustain ischemia reperfusion injury during the procedure. As described in more detail in Example 12, reduced Sg1P levels were detected in patients following cardiac bypass surgery and lower blood Sg1P levels in patients correlated with worse post-operative outcome.

We demonstrated that exogenous Sg1P significantly reduced renal and hepatic vascular endothelial cell injury in mice subjected to liver IR and protected human endothelial cell cultures exposed to apoptosis-inducing conditions (see Examples 5 and 8). Accordingly, in another embodiment, the present invention relates to a method for reducing, inhibiting or preventing endothelial cell injury, the method comprising administering an effective amount of sphinganine-1-phosphate. The endothelial cell injury may be associated with apoptosis, necrosis or inflammation. In another embodiment, the present invention relates to a method for reducing, inhibiting or preventing endothelial cell injury. In certain embodiments, the endothelial cell may be from endothelial cells of the liver, lung, lymph node, umbilical vein, brain, intestine, and pancreas.

### III. Diagnostic Uses of Sphinganine-1-Phosphate

The present invention relates to the discovery that significantly lower Sg1P levels were detected in blood samples from mice and humans subjected to liver transplantation and that the Sg1P levels correlated with patient outcome. A bigger post-operative drop in serum Sg1P correlated with worse outcome. Thus, Sg1P levels may be used as a diagnostic for monitoring the renal health of liver transplantation patients as well as a predictor of transplantation success and patient outcome/mortality.

In one embodiment, the invention relates to a method of identifying a mammalian subject who is developing renal failure due to an ischemia reperfusion injury comprising the steps of determining the concentration of sphinganine-1-phosphate in a biological sample from said subject who has suffered an ischemia reperfusion injury; comparing the concentration of sphinganine-1-phosphate in the subject with a reference concentration of sphinganine-1-phosphate; wherein reduced sphinganine-1-phosphate concentration in the subject compared to the reference concentration is indicative that the subject is developing renal failure. In certain embodiments of the invention, the reference concentration is the concentration in a biological sample from at least one subject who has not suffered an ischemia reperfusion injury. In certain embodiments of the invention, the reference concentration is the concentration of sphinganine-1-phosphate in a biological sample from the subject prior to suffering from the ischemia reperfusion injury. In one embodiment, the ischemia reperfusion injury is associated with organ transplant, liver ischemia, kidney ischemia, stroke, abdominal aortic occlusion, abdominal aortic bleeding or perioperative ischemia due to cardiopulmonary bypass surgery. In some embodiments, the organ transplant is selected from the group consisting of a liver transplant, a kidney transplant, a heart transplant, a lung transplant, a pancreas transplant and an intestine transplant.

In certain embodiments, a reduction in Sg1P concentration/level to <50% of the reference concentration is indicative of renal failure developing in the subject. In certain embodiments, a reduction in Sg1P concentration/level to <45% of the reference concentration is indicative of renal failure developing in the subject. In certain embodiments, a reduction in Sg1P concentration/level to <40% of the reference concentration is indicative of renal failure developing in the subject. In certain embodiments, a reduction in Sg1P concentration/level to <35% of the reference concentration is indicative of renal failure developing in the subject. In certain embodiments, a reduction in Sg1P concentration/level to <30% of the reference concentration is indicative of renal failure developing in the subject. In certain embodiments, a reduction in Sg1P concentration/level to <25% of the reference concentration is indicative of renal failure developing in the subject. In certain embodiments, a reduction in Sg1P concentration/level to <20% of the reference concentration is indicative of renal failure developing in the subject. In certain embodiments, a reduction in Sg1P concentration/level to <15% of the reference concentration is indicative of renal failure developing in the subject. In certain embodiments, a reduction in Sg1P concentration/level to <10% of the reference concentration is indicative of renal failure developing in the subject.

Subjects that may benefit from the methods of the present invention are those that are developing renal failure due to an ischemia reperfusion injury, or those that are at risk for developing renal failure due to an ischemia reperfusion injury.

The biological samples used in the methods described herein may comprise cells from the eye, ear, nose, throat, teeth, tongue, epidermis, epithelium, blood, tears, saliva, mucus, urinary tract, urine, muscle, cartilage, skin, or any other tissue or bodily fluid from which sufficient DNA, RNA or protein can be obtained. In certain embodiments, the biological sample is selected from the group consisting of blood, urine, saliva, tissue sample from a liver, tissue sample from a kidney, tissue sample from a heart, tissue sample from a muscle and tissue sample from a blood vessel.

This invention will be better understood from the Experimental Details which follow. However, one skilled in the art will readily appreciate that the specific methods and results discussed are merely illustrative of the invention as described more fully in the embodiments which follow thereafter.

### EXPERIMENTAL DETAILS

### Example 1

### Murine Model of Liver Ischemia Reperfusion (Hepatic IR)

Mice were anesthetized with intraperitoneal pentobarbital (50 mg/kg or to effect). Mice were placed under a heating lamp and on a 37° C. heating pad. After a midline laparotomy and intraperitoneal application of 20 U heparin, left lateral and median lobes of the liver were subjected to ischemia with a microaneurysm clip occluding the hepatic triad above the bifurcation. This method of partial hepatic ischemia results in a segmental (˜70%) hepatic ischemia but spares the right lobe of the liver and prevents mesenteric venous congestion by allowing portal decompression throughout the right and caudate lobes of the liver. The liver was then repositioned in the peritoneal cavity in its original location for 60 minutes. The liver was kept moist with gauze soaked in 0.9% normal saline. The body temperature was monitored by an infrared temperature sensor (Linear Laboratories, Fremont, Calif.) every 10 min. and maintained at 37° C. using a heating lamp and a heating pad. After 60 minutes, the liver was reperfused and the wound closed. Sham-operated mice were subjected to laparotomy and identical liver manipulations without vascular occlusion. Two, 4 and 24 hr after reperfusion, plasma was collected for the measurement of alanine aminotransferase (ALT), a common indicator of liver damage, as well as creatinine (Cr) a common indicator of kidney damage. The liver tissue subjected to IR was collected to measure percent liver necrosis (2, 4 and 24 hr after IR), neutrophil infiltration (with immunohistochemistry, 4 and 24 hr after IR), apoptosis (with terminal deoxynucleotidyl transferase-mediated dUTP nick end labeling staining, caspase 3 immunoblotting and DNA laddering, 24 hr after IR), and inflammation by RT-PCR for pro-inflammatory mRNAs (4 hr after IR).

### Example 2

### Murine Model of Renal and Hepatic Failure Following Liver IR

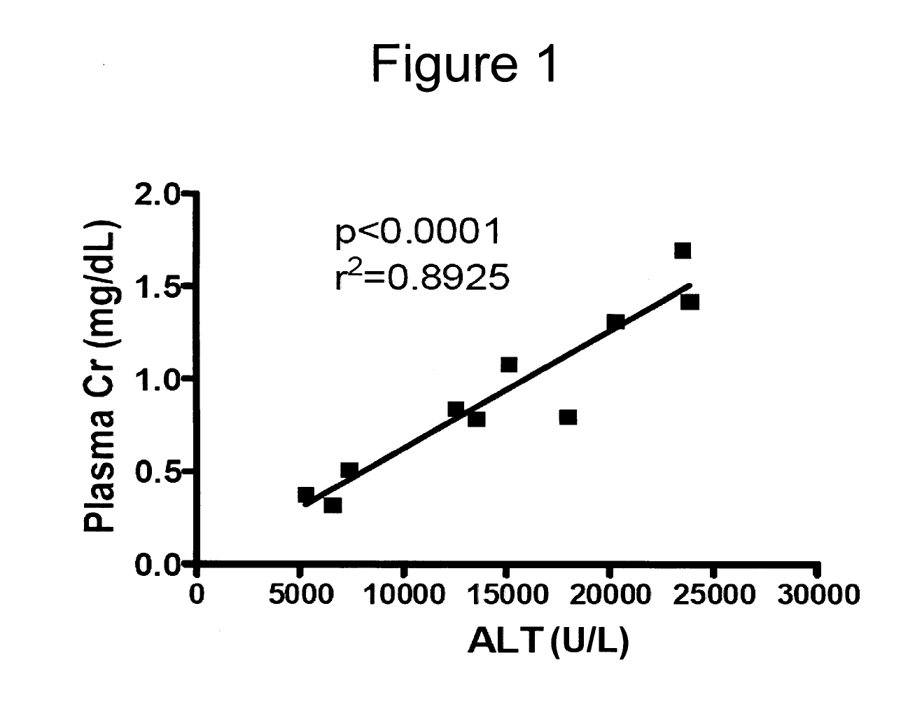

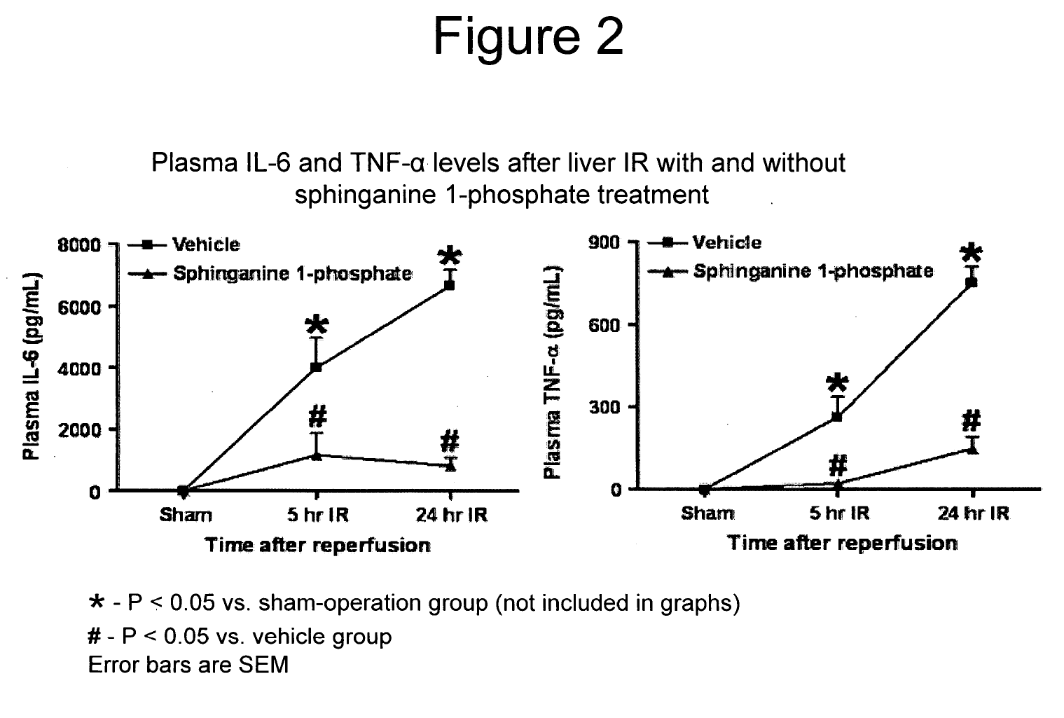

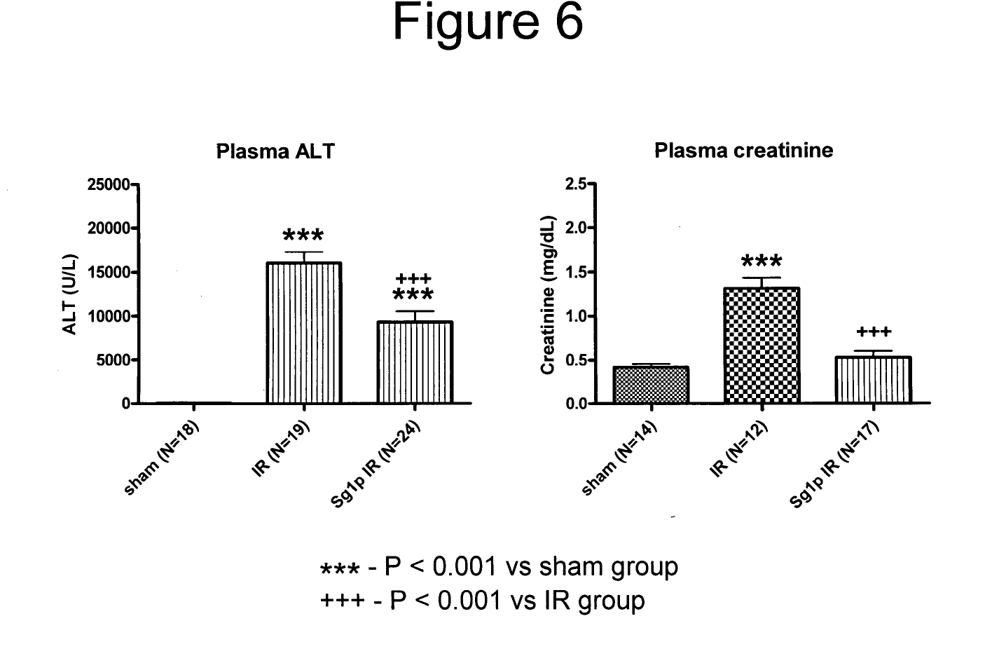

Preliminary studies showed that C57BL/6 mice subjected to 60 min. of ˜70% liver ischemia and 24 hr reperfusion (liver IR) developed severe liver dysfunction with significantly elevated plasma ALT levels (15076±1174 U/L, N=6, p<0.0005 vs. sham-operated mice: ALT=72±9 U/L, N=6). See FIG. 6, left and Table 3. Moreover, C57BL/6 mice subjected to liver IR also developed acute kidney injury (AKI) with a rise in plasma creatinine (Cr) levels 24 hr after liver IR (Cr=1.08±0.07 mg/dL, N=6, p=0.01 vs. sham-operated mice: Cr=0.43±0.03 mg/dL, N=6). See FIG. 6, right and Table 2. As shown in FIG. 1, there was a correlation between the severity of liver dysfunction (ALT) and the degree of AKI (Cr) 24 hr after IR (p<0.0001 and r²=0.8925). Liver IR injury in mice also increased the plasma levels of TNF-α (852±93 pg/ml plasma, N=5 vs. undetectable levels for sham-operated mice, N=5), a cytokine implicated in multiorgan dysfunction after liver IR (Tsung et al., 2005), as well as plasma levels of interleukin 6 (IL-6). See FIG. 2.

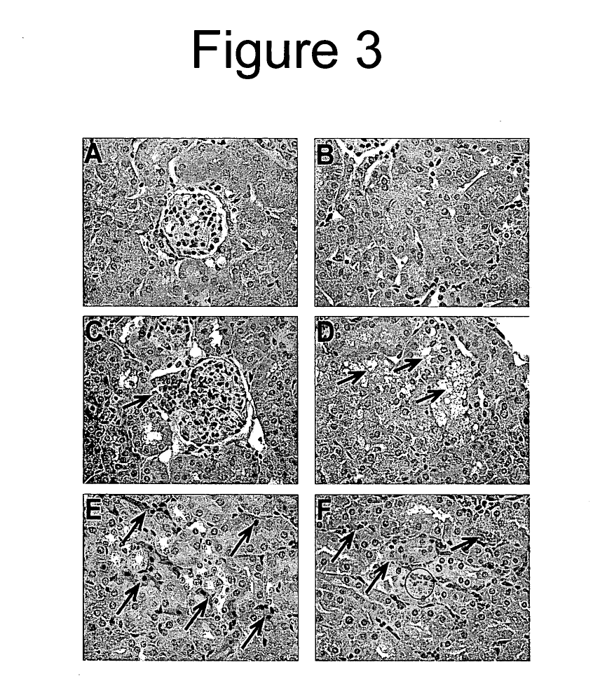

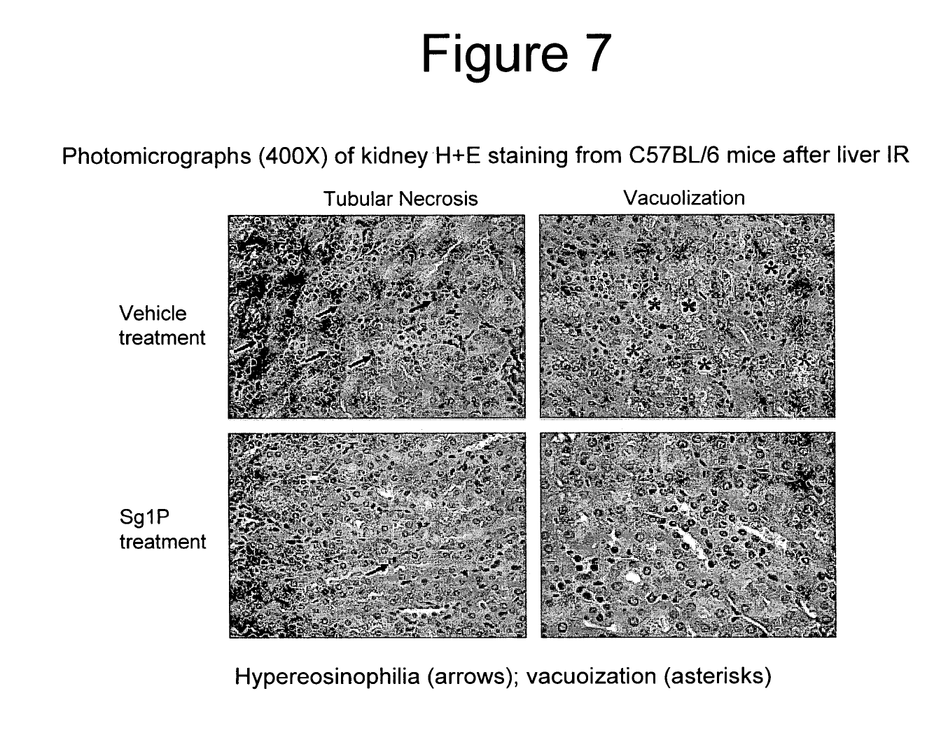

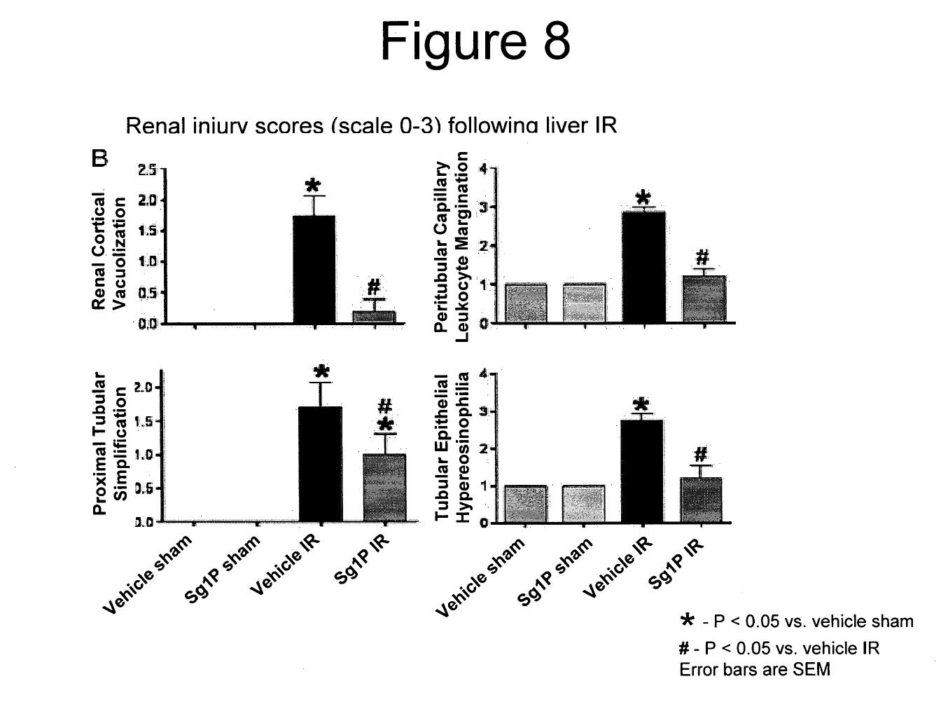

Liver IR-induced AKI in mice shared similar histological changes observed in human AKI associated with liver failure, as observed in H&E slides of kidney sections taken from the mice (see FIG. 3). Multifocal acute tubular injury including individual cell necrosis involving the juxtamedullary proximal tubules of the S3 segment was observed. In addition, the cortical tubules exhibited focal tubular simplification, cytoplasmic vacuolization, dilated lumina and focal granular bile/heme casts, accompanied by mild interstitial edema (see FIGS. 7 and 8). Endothelial apoptosis of peritubular capillaries was also visible in H&E sections as linear arrays of apoptotic bodies corresponding to the interstitial capillary lining endothelium. Cells in the S3 segment of proximal tubules showed a significantly higher number of necrotic cells compared to the sham-operated mice. The number of glomeruli with detectable juxtaglomerular apparatus and the number of cells per juxtaglomerular apparatus were both increased after liver IR, indicating hyperplasia of the juxtaglomerular apparatus. H&E staining also revealed extensive liver damage following liver IR. Liver IR produced large necrotic areas of liver tissue. The average percentage of necrotic areas in liver samples taken from liver IR mice were 92%±2% (n=6), while no necrosis was found in sham-operated mice. Livers were also analyzed for the degree of hepatocellular damage. The ischemic lobes in the control group showed severe hepatocyte vacuolization, necrosis, and sinusoidal congestion.

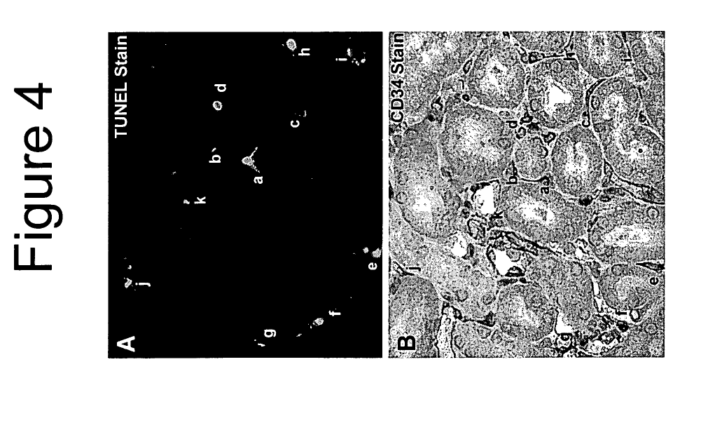

In addition, mice subjected to liver IR displayed marked renal endothelial apoptosis (TUNEL positive cells, data not shown). TUNEL positive cells were heavily localized to the perivascular area rather than to the renal proximal tubule cells (cells typically showing apoptotic changes after renal IR). TUNEL positive cells were confirmed to be endothelial cells by staining parallel kidney sections with TUNEL and CD34 (an endothelial cell marker) and confirming that TUNEL positive cells also stained for CD34, as shown in FIG. 4.

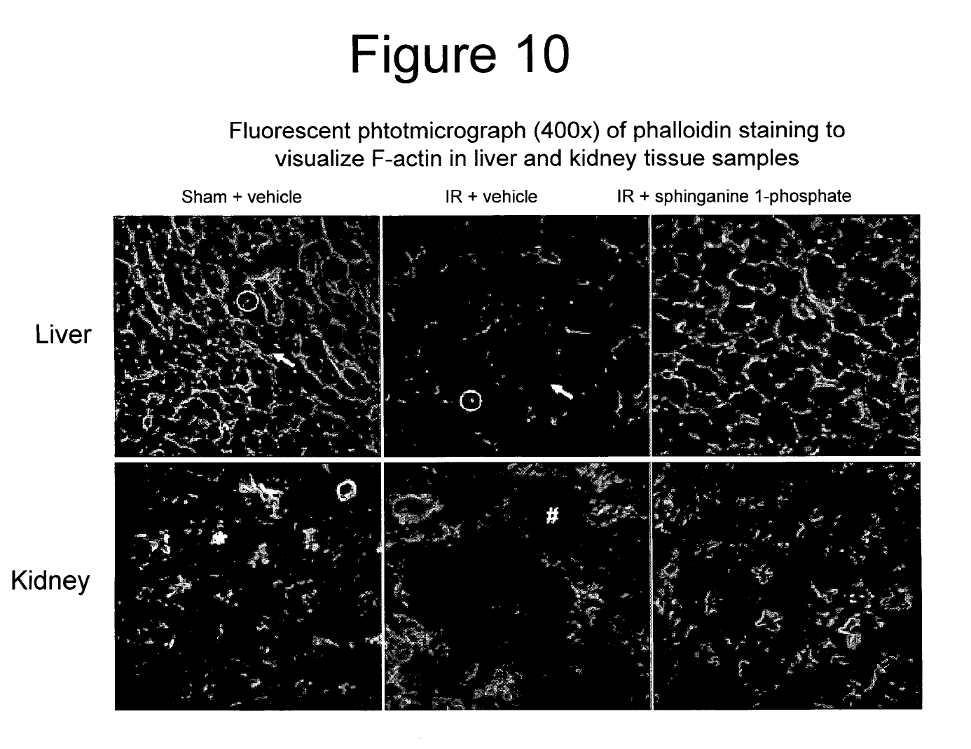

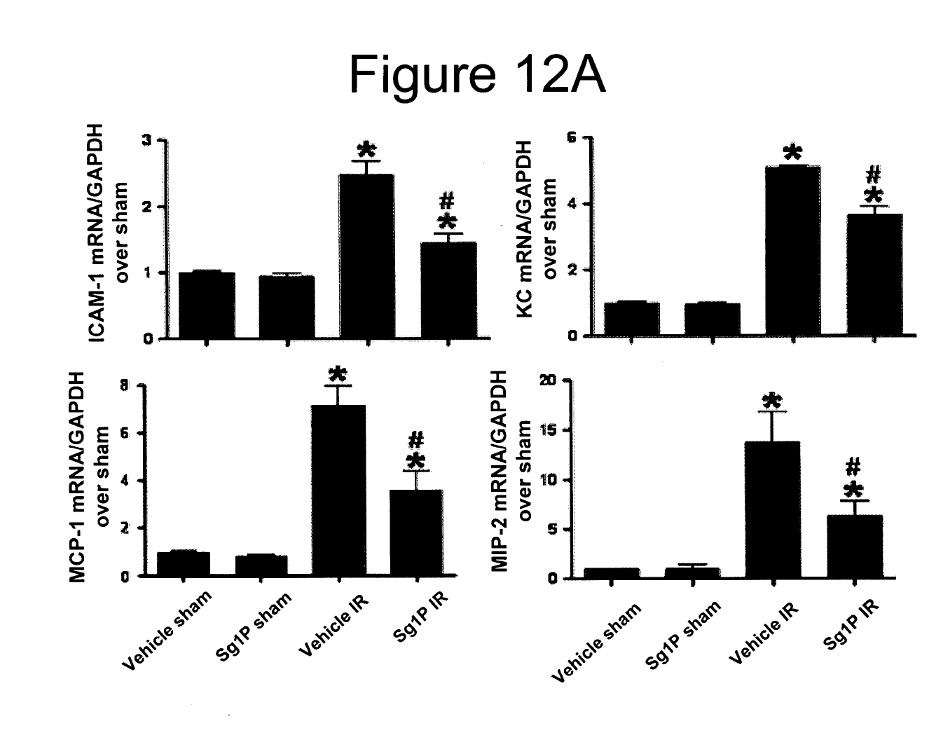

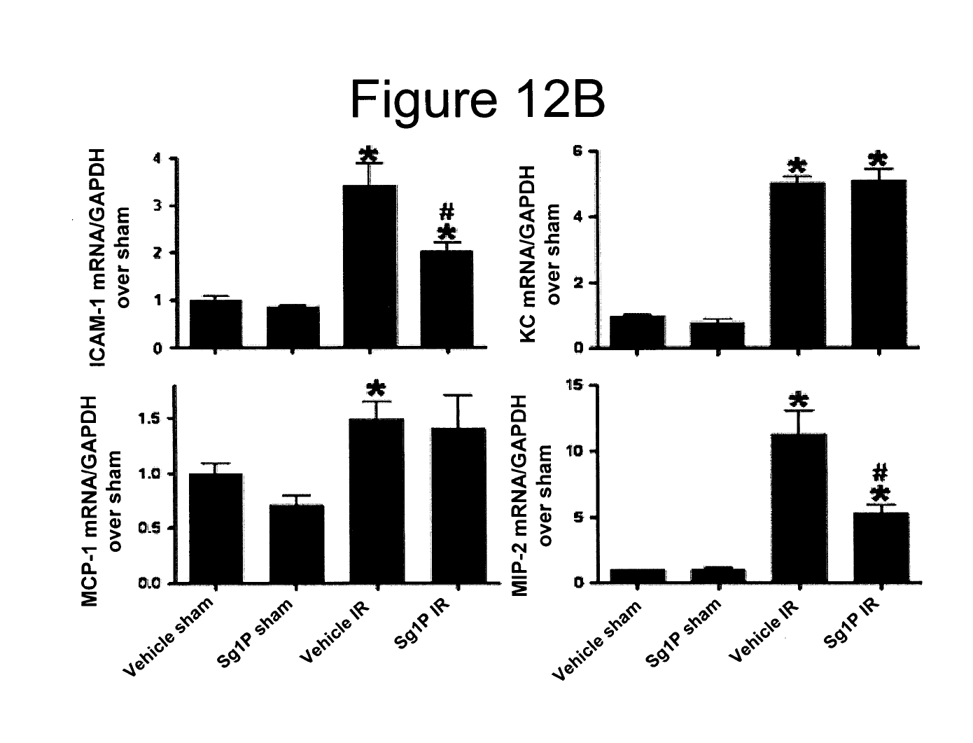

Liver IR injury also resulted in a severe loss of F-actin in renal proximal tubules and in liver basolateral membranes and bile canalicuar membranes (phalloidin stain and immunoblotting, see FIG. 10). Loss of F-actin promotes renal cells to undergo apoptosis (Genesca et al., 2006). In addition, liver IR also caused renal pro-inflammatory mRNA expression (such as ICAM-1, KC, MCP-1 and MIP-2 mRNA) in both liver (FIG. 12A) and kidney (FIG. 12B). Liver IR also caused infiltration of polymorphonuclear neutrophils into the kidney and liver.

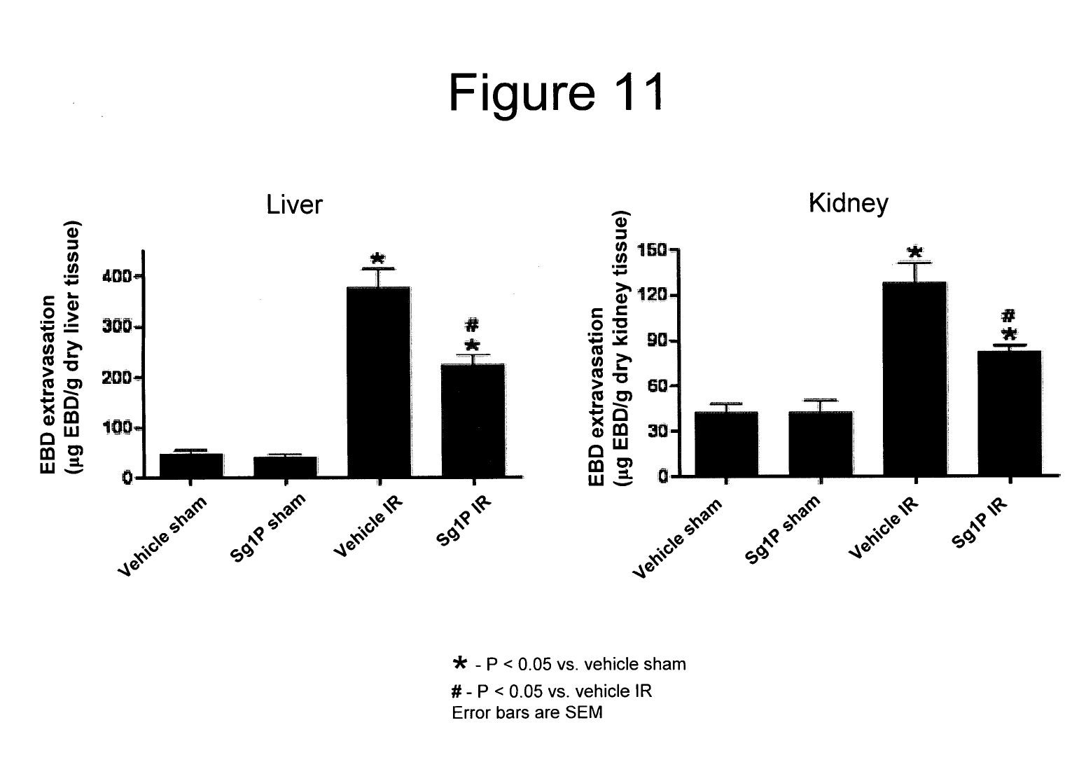

After observing marked endothelial apoptosis following liver IR, liver and kidney vascular permeability after liver IR was measured with Evans blue dye (EBD) injection. EBD binds to plasma proteins and its appearance in extravascular tissues reflects an increase in vascular permeability, which is indicative of organ injury. Liver IR increased the EBD content (in μg EBD/g dry weight) in the liver (377±48, N=6) compared to sham-operated mice (48±7, N=4, p<0.001). See FIG. 11, left. Liver IR also increased the EBD content in the kidney as well (liver IR: 84±6, N=5 vs. sham: 42±5, N=4, p<0.02). See FIG. 11, right.

### Example 3

### Serum Sg1P Levels Drop after Liver IR in Mice

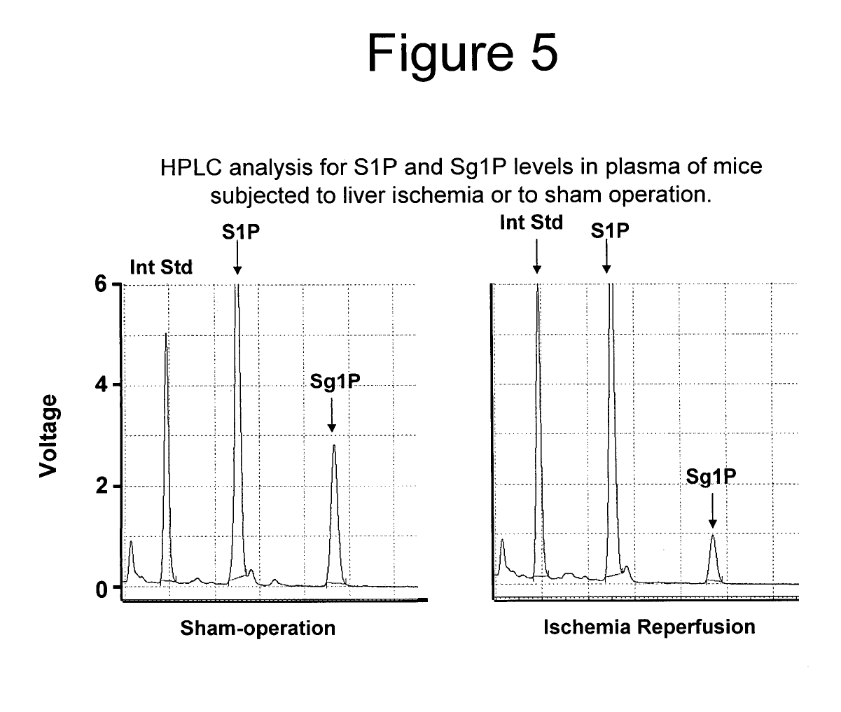

It was postulated that depletion of sphingosine-1-phosphate (S1P), an endogenous molecule well known to protect against endothelial dysfunction, may explain the increased renal endothelial damage after liver IR. Therefore, plasma levels of S1P (in pmol/μl plasma) were measured in mice after liver IR or sham-operation utilizing HPLC. Quantitative analysis using HPLC to measure S1P levels was be performed as previously described by Min et al. (2002) with enzymatic dephosphorylation of S1P by alkaline phosphatase and subsequent analysis of o-phthalaldehyde derivatives of the liberated sphingosine bases. As shown in FIG. 5, plasma levels of S1P were not decreased in mice subjected liver IR as compared to sham-operation. However, HPLC analysis revealed that another sphingolipid metabolite, sphinganine-1-phosphate (Sg1P), was decreased following liver IR (see FIG. 5).

Additional experiments summarized in Table 1 below further confirmed that plasma levels of S1P did not change in mice following liver IR compared to sham-operated controls (0.978±0.088 fold sham, N=11, p=0.785). However, plasma levels of Sg1P decreased significantly in mice subjected to liver IR compared to the sham-operated mice (0.438±0.114 fold sham, N=8, p<0.0001.

TABLE 1
Serum Sg1P and S1P levels 24 hours post-liver IR

| | C57BL6 Mice - Sg1P | | | | | | |
|---|---|---|---|---|---|---|---|
| | Level (Fold Sham) | | | | C57BL6 Mice - S1P | | |
| | | | IR + | IR + | Level (Fold Sham) | | |
| Sg1P | Sham | IR | SG1P | S1P | S1P | Sham | IR |
| Average | 1.000 | 0.438 | 0.977 | 1.041 | Average | 1.001 | 0.978 |
| SE | 0.053 | 0.114 | 0.187 | 0.149 | SE | 0.039 | 0.088 |
| N | 21 | 8 | 14 | 9 | N | 23 | 11 |
| P vs | | 1 × 10⁵ | 0.891 | 0.746 | P vs | | 0.785 |
| Sham | | | | | Sham | | |
| P vs IR | | | 0.046 | 0.004 | | | |

Not only were plasma levels of Sg1P decreased following liver IR but renal levels of Sg1P were also reduced after liver IR (20±2 fmol/mg protein, N=4) compared to sham-operated mice (40±2 fmol/mg protein, N=4). Renal levels of S1P (as with serum S1P levels) did not change after liver IR (610±135 fmol/mg protein, N=4) vs. sham-operation (568±167 fmol/mg protein, N=4). Therefore, following liver IR, there was a selective reduction in Sg1P levels in the plasma and kidney, suggesting that the reduction in Sg1P may have contributed to renal dysfunction seen after liver IR.

### Example 4

### Administration of Sg1P Protects Against Kidney and Liver Injury Following Liver IR in Mice

To determine if the selective reduction in Sg1P contributed to renal dysfunction following liver IR and whether exogenous Sg1P improves renal and hepatic dysfunction after liver IR, mice subjected to liver IR were administered Sg1P. The mice were injected with 0.1 mg/kg Sg1P intravenously (i.v.) 15 minutes before and 0.2 mg/kg subcutaneously (s.c.) 2 hours after liver reperfusion (Sg1P treatment). Control liver IR mice were also subjected to liver IR, but received treatment with vehicle only (4 mg/ml fatty acid-free bovine serum albumin solution). Sg1P treatment helped maintain the serum concentration of Sg1P in mice subjected to liver IR at the same level as sham-operated mice. Whereas plasma Sg1P went down to 0.438±0.114 fold sham 24 hours following liver IR, Sg1P treatment completely restored plasma Sg1P levels to (0.977±0.187 fold sham). See Table 1.

In mice subjected to liver IR, administration of Sg1P also provided significant protection against renal injury. As described above, Creatinine levels measured 24 hours after liver IR (w/vehicle administration) was higher compared to sham-operated controls, indicating renal injury. However, with Sg1P treatment, the rise in creatinine levels following liver IR was significantly suppressed. See FIG. 6, top right and Table 2.

TABLE 2
Serum Creatinine: Kidney function in mice subjected to liver IR

| | Mean Creatinine | | |
|---|---|---|---|
| | (mg/dL) | S.E. | N |
| Sham (C57BL/6) | 0.41 | 0.04 | 16 |
| Sham + Vehicle | 0.43 | 0.03 | 6 |
| Sham + Sg1P | 0.46 | 0.05 | 6 |
| Hepatic IR + Vehicle | 1.08 | 0.07 | 6 |
| Hepatic IR + Sg1P | 0.55 | 0.05 | 6 |
| Hepatic IR + S1P | 0.72 | 0.13 | 9 |

In addition, H&E slides of kidney from liver IR mice treated with Sg1P showed significantly less indications of AKI (renal cortical vacuolization, peritubular capillary leukocyte margination, proximal simplification, and tubular hypereosinophilia) compared to vehicle-treated controls (FIGS. 7 and 8).

Sg1P treatment also provided protection against liver injury caused by liver IR. Sg1P treatment significantly suppressed the rise of serum ALT following liver IR. See FIG. 6, top left and Table 3.

TABLE 3
Serum ALT: Liver function in mice subjected to liver IR

| | Mean ALT (U/L) | S.E. | N |
|---|---|---|---|
| Sham (C57BL/6) | 61 | 30 | 17 |
| Sham + Vehicle | 72 | 9 | 6 |
| Sham + Sg1P | 80 | 6 | 6 |
| Hepatic IR + Vehicle | 15076 | 1174 | 6 |
| Hepatic IR + Sg1P | 7474 | 557 | 6 |
| Hepatic IR + S1P | 9178 | 1822 | 9 |

H&E slides of liver from mice subjected to hepatic IR showed large necrotic areas. Correlating with significantly improved hepatic function, reduced liver necrosis was observed in liver IR mice treated with sphinganine 1-phosphate. The average percent necrotic areas for vehicle-treated mice were 92±2% (N=6) and sphinganine 1-phosphate-treatment reduced this percent necrosis to 44±8% (N=7, P<0.05). We failed to detect necrosis in liver sections from sham-operated mice. Livers were also analyzed for the degree of hepatocellular damage using the Suzuki's criteria (Suzuki et al., Transplantation 1993, Vol. 55, pp. 1265-1272). To obtain a Suzuki score, 3 liver injury indices are graded: sinusoidal congestion (0-4), hepatocyte necrosis (0-4), and ballooning degeneration (0-4) are graded for a total score of 0-12. No necrosis, congestion, or centrilobular ballooning is given a score of 0 whereas severe congestion/ballooning and >60% lobular necrosis is given a value of 4. The ischemic lobes in the control group showed severe hepatocyte vacuolization, necrosis and sinusoidal congestion (Suzuki score=8.7±0.3, N=5). Mice treated with sphinganine 1-phosphate revealed significantly less necrosis/sinusoidal congestion and better preservation of lobular architecture (Suzuki score=5.2±0.8, N=5, P<0.01). See Table 7.

The infiltration of polymorphonuclear neutrophils into the kidney and liver caused by liver IR was significantly suppressed by Sg1P treatment. The number of neutrophils present in the necrotic areas of the liver samples taken from mice subjected to liver IR was lower when the mice received Sg1P treatment (21.8±7.1 neutrophils/field, n=6) compared to vehicle-only treatment (39.5±11.8 neutrophils/field, n=6). The number of neutrophils present was also lower in the kidneys of Sg1P-treated mice (0.8±0.3 neutrophils/field, n=5) compared to vehicle-treated ones (14.9±3.1 neutrophils/field, n=5)

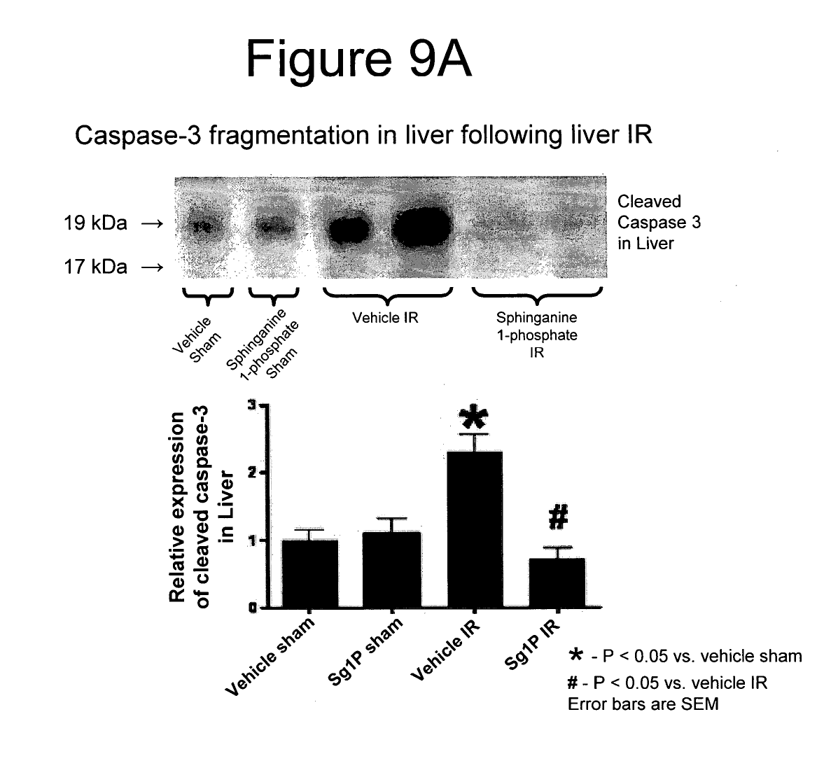

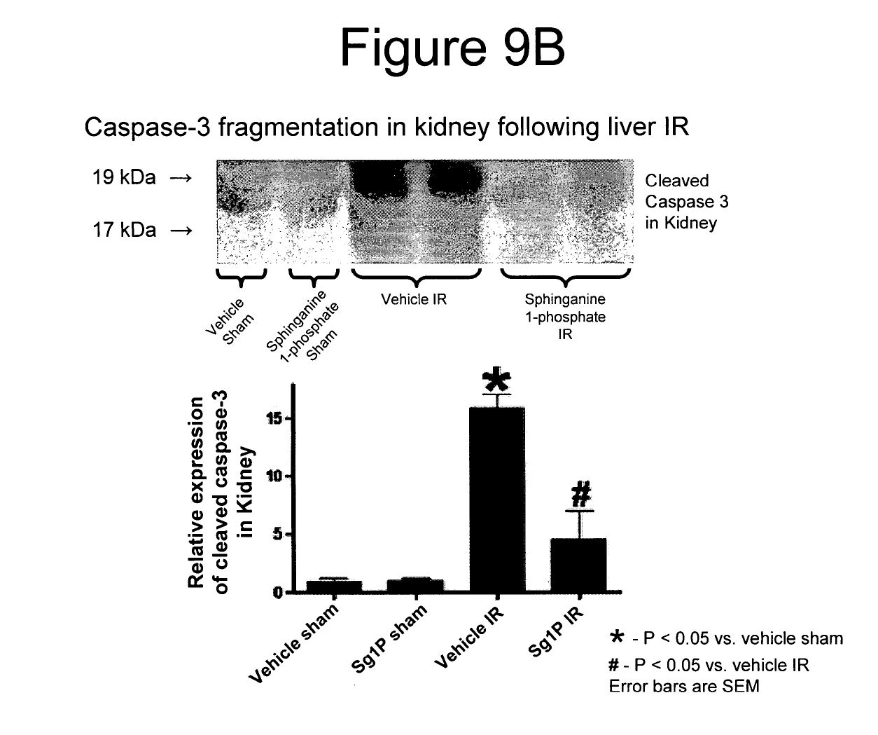

Apoptosis was detected in both liver and kidney taken from mice subjected to liver IR, through TUNEL staining, DNA laddering and caspase 3 fragmentation. Sg1P treatment significantly reduced TUNEL staining, DNA laddering in both liver and kidney. Sg1P treatment also significantly reduced caspase 3 fragmentation in both liver (FIG. 9A) and kidney (FIG. 9B). Liver IR injury also resulted in a severe loss of F-actin in renal proximal tubules and in liver basolateral membranes and bile canalicuar membranes (phalloidin stain and immunoblotting). In liver and kidney of mice subjected to liver IR, Sg1P treatment resulted in better-preserved F-actin structure compared to vehicle treatment. See FIG. 10.

Sg1P also suppressed the increase in vascular permeability normally induced in both liver and kidney after liver IR. Liver IR increased the Evans blue dye (EBD) extravasation (a measure of vascular permeability) in the liver and kidney in both Sg1P- and vehicle-treated groups, but the increase in EBD content was significantly lower for the Sg1P-treated mice compared with the vehicle-treated group. See FIG. 11.

Liver IR resulted in increased expression of mRNA encoding pro-inflammatory proteins (such as ICAM-1, KC, MCP-1 and MIP-2) in both liver and kidney. Sg1P treatment suppressed this increase in both liver and kidney. The extent of the suppression was different between kidney and liver. Sg1P treatment suppressed the liver IR-induced upregulation of all 4 mRNAs encoding pro-inflammatory proteins in the liver (FIG. 12A). In kidney, Sg1P treatment suppressed the liver IR-induced ICAM-1 and MIP-2 upregulation, but not for KC or MCP-1 (FIG. 12B).

As described above, liver IR injury in mice increased the plasma levels of TNF-α (852±93 pg/ml plasma, N=5 vs. undetectable levels for sham-operated mice, N=5), a cytokine implicated in multiorgan dysfunction after liver IR (Tsung et al., 2005), as well as plasma levels of interleukin 6 (IL-6). Sg1P treatment almost eliminated the liver IR-induced increase in TNF-α and IL-6. See FIG. 2.

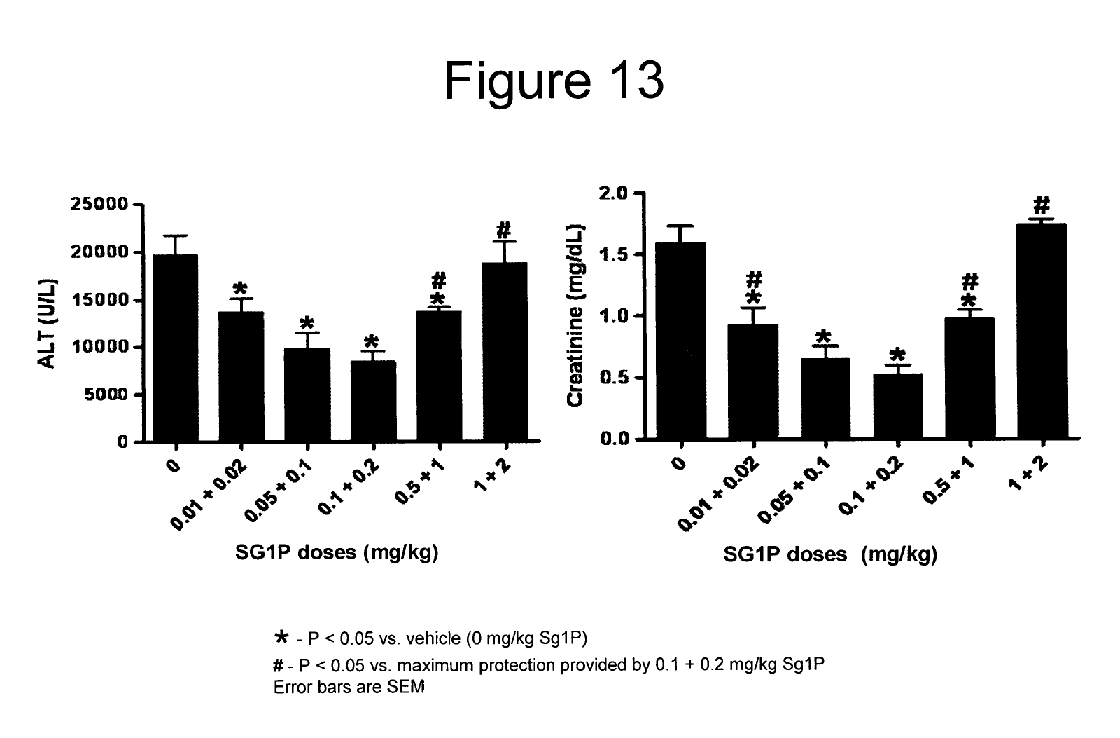

Further experiments were conducted to determine a Sg1P dose response curve for maximal reduction of kidney and liver failure resulting from liver IR. The doses tested were the following (N=6-17): (1) vehicle only; (2) 0.01 mg/kg intravenous (i.v.) prior to reperfusion followed by 0.02 mg/kg subcutaneous (s.c.) 2 hours after reperfusion; (3) 0.05 mg/kg i.v. prior to reperfusion followed by 0.1 mg/kg s.c. 2 hours after reperfusion; (4) 0.1 mg/kg i.v. prior to reperfusion followed by 0.2 mg/kg s.c. 2 hours after reperfusion; (5) 0.5 mg/kg i.v. prior to reperfusion followed by 1.0 mg/kg s.c. 2 hours after reperfusion; and (6) 1.0 mg/kg i.v. prior to reperfusion followed by 2.0 mg/kg s.c. 2 hours after reperfusion. As shown in FIG. 13, the dosage of 0.1 mg/kg i.v. prior to reperfusion followed by 0.2 mg/kg s.c. 2 hours after reperfusion was found to be the most effective dose for treating both kidney and liver failure, as measured by serum creatinine and ALT levels, respectively.

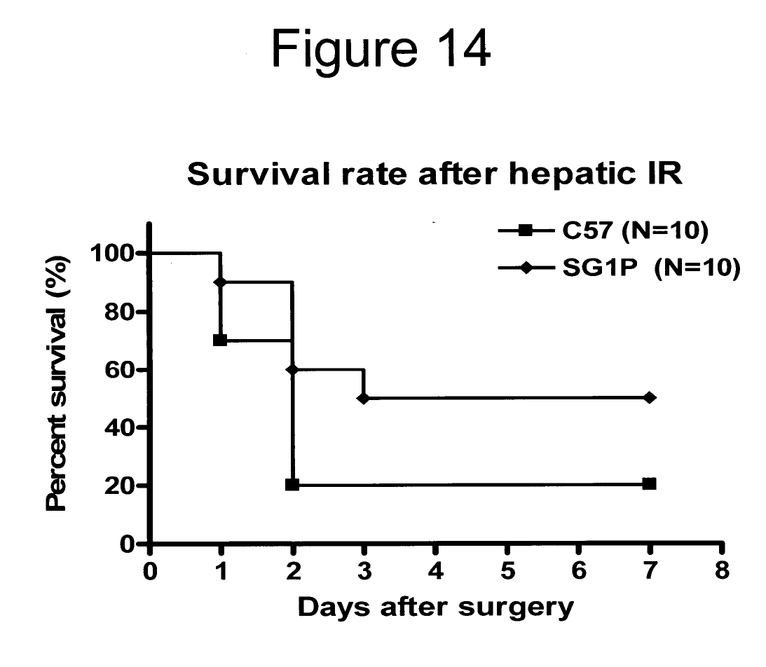

The survival of the mice post-liver IR was also higher in mice treated with Sg1P, as shown in FIG. 14. Without Sg1P administration, 20% of mice subjected to liver IR stayed alive after 1 week. With Sg1P treatment, 50% of mice subjected to liver IR that received Sg1P remained alive after the same time period.

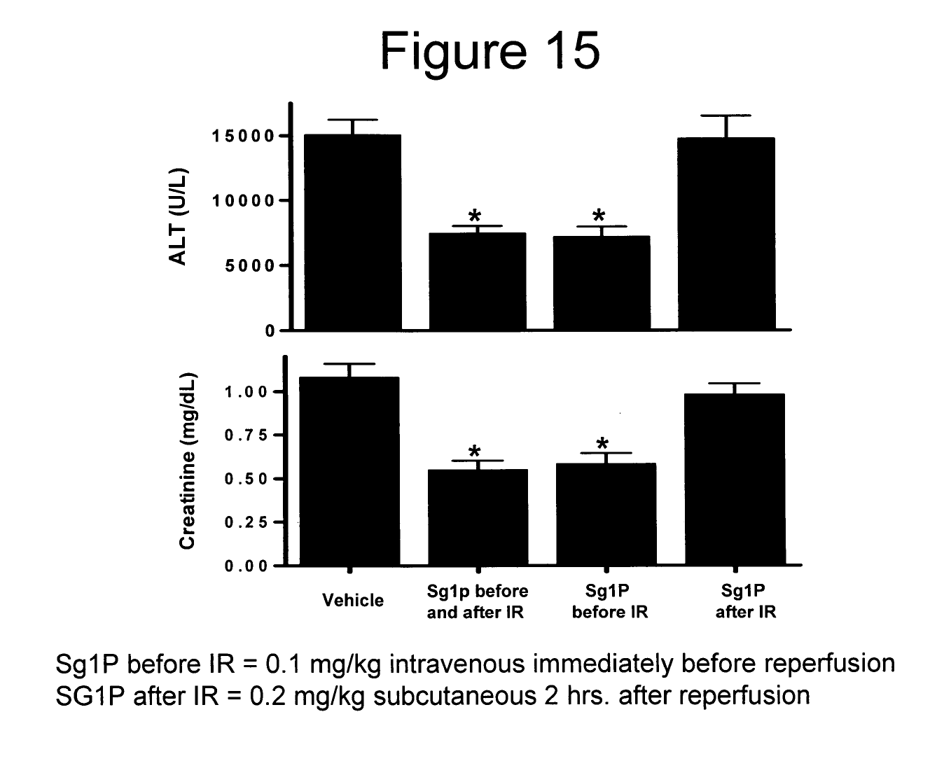

The timing of Sg1P administration in relation to liver IR was also investigated. The effect of a single dose of Sg1P, given immediately before reperfusion (0.1 mg/kg, i.v.) or two hours after reperfusion (0.2 mg/kg s.c.) was compared. We found that Sg1P administered before reperfusion was protective (ALT=7197±753 U/L, N=6 and Cr=0.58±0.06 mg/dL, N=6) whereas the dose given 2 hrs after reperfusion was not protective (ALT=14762±1732 U/L, N=6 and Cr=0.98±0.06 mg/dL, N=6). See FIG. 15. Similar results were obtained when the 2 hour post-reperfution dosage of Sg1P was administered i.v., rather than s.c. Intravenous administration of 0.2 mg/kg Sg1P 2 hours post-reperfusion resulted in attenuated liver protection (12063±593 U/L; N=4; p=0.03 vs. vehicle) and no kidney protection (0.93±0.05 mg/dL; N=4; p=0.01 vs. vehicle).

In summary, Sg1P treatment was shown to be effective in protecting kidney and liver from liver IR-related damage through a variety of assays: biochemical assays of organ function (serum creatinine and ALT); histological analyses of tissue integrity, necrosis, infiltration of neutrophils, and apoptosis; detection of pro-inflammatory molecules; assay of organ vascular permeability (EBD extravasation assay); and mouse survival. In all assays, Sg1P was shown to promote organ protection in mice subjected to liver IR.

Sphinganine, sphingosine-1-phosphate, and sphingosine, were also effective in treating organ failure related to liver IR. Sphinganine, the unphosphorylated form of Sg1P, provided protection from liver IR-induced renal failure. In mice subjected to liver IR, sphinganine treatment (0.1 mg/kg i.v. prior to ischemia and 0.2 mg mg·kg 2 hrs. after reperfusion) resulted in a serum creatinine level of 0.58±0.05 mg/dL (N=8), which is significantly lower than vehicle treatment and similar to Sg1P treatment. Sphinganine treatment also protected liver function. Serum ALT in mice subjected to liver IR and treated with sphinganine was 7170±1282 U/L (N=8). Similar results were obtained with S1P treatment (0.1 mg/kg i.v. before reperfusion and 0.2 mg/kg s.c. 2 h after reperfusion), in which serum creatinine 24 hours following liver IR was 0.72±0.13 mg/dL (N=9) and serum ALT was 9178±1822 U/L (N=9), as well as with sphingosine, in which serum creatinine 24 hours following liver IR was 0.76±0.06 mg/dL (N=6) and serum ALT was 9269±583 U/L (N=6). For a summary of the results, see Table 4. The effectiveness of sphinganine, S1P and sphingosine treatments is based on their ability to restore serum Sg1P. S1P treatment in mice subjected to liver IR completely rescued serum Sg1P levels to levels equal to control mice subjected only to sham operations (see Table 1). Similar rescues of serum Sg1P levels were also seen in mice treated with sphinganine and sphingosine (data not shown).

TABLE 4
Sphinganine, S1P and Sphingosine treatment: Kidney/liver function

| | Creatinine (mg/dL) | | | Mean ALT (U/L) | | |
|---|---|---|---|---|---|---|
| 24 h post-IR | Mean | S.E. | N | Mean | S.E. | N |
| Sham + | 0.43 | 0.03 | 6 | 72 | 9 | 6 |
| Vehicle | | | | | | |
| Hepatic IR + | 1.08 | 0.07 | 6 | 15076 | 1174 | 6 |
| Vehicle | | | | | | |
| Hepatic IR + | 0.55 | 0.05 | 6 | 7474 | 557 | 6 |
| Sg1P | | | | | | |
| Hepatic IR + | 0.58 | 0.05 | 8 | 7170 | 1282 | 8 |
| Sphinganine | | | | | | |
| Hepatic IR + | 0.72 | 0.13 | 9 | 9178 | 1822 | 9 |
| S1P | | | | | | |
| Hepatic IR + | 0.76 | 0.06 | 6 | 9269 | 583 | 6 |
| Sphingosine | | | | | | |

### Example 5

### The Therapeutic Action of Sg1P Administration in Reducing Liver and Renal Failure Due to Liver IR is Mediated by the S1P Receptor Type 1

We demonstrated that the therapeutic action of Sg1P administration in reducing renal and hepatic failure due to liver IR is mediated by the S1P receptors, in particular S1P receptor type 1 (S1P1 receptor; see Table 5).

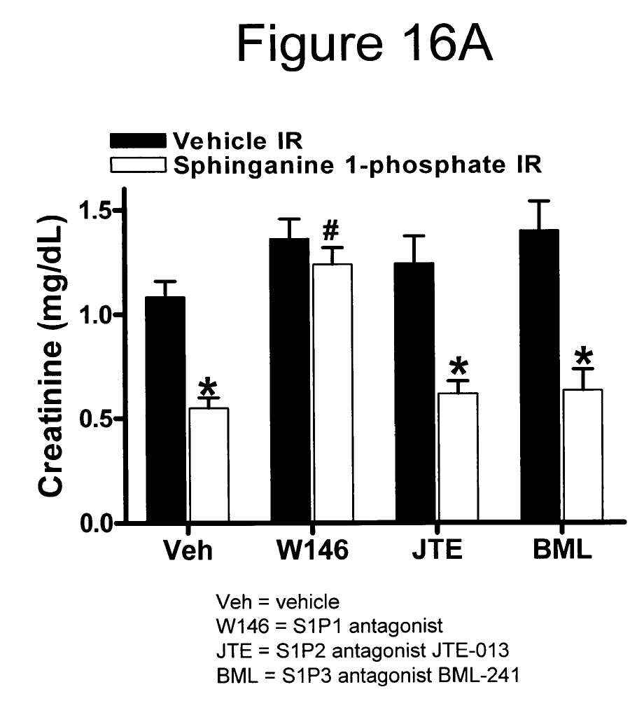

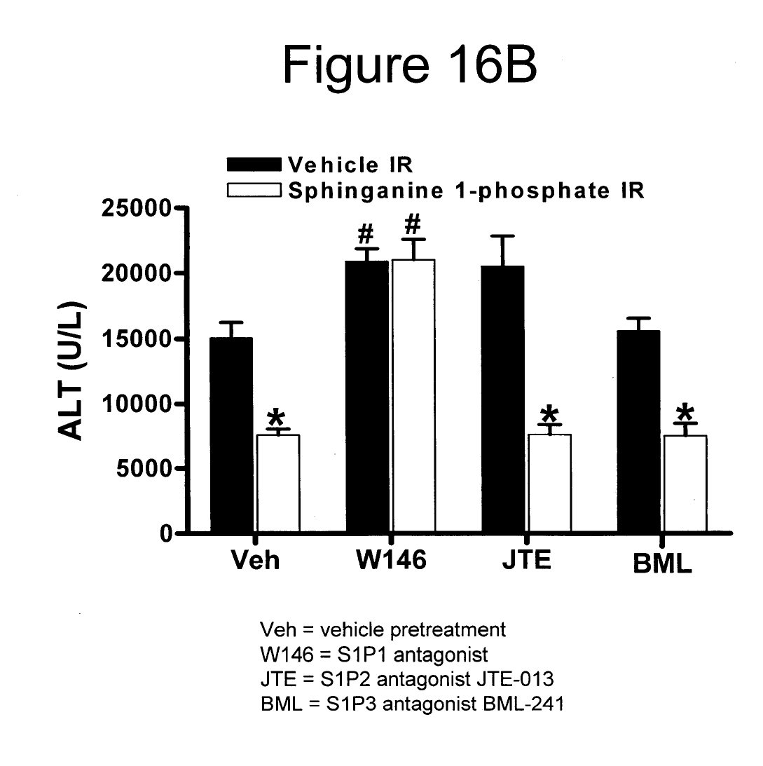

When preceded by the administration of VPC 23019 (a pharmacological antagonist of S1P1 and S1P3 receptors, but 50-fold more selective for S1P1), Sg1P treatment failed to reduce renal injury induced by liver IR (as measured by serum Cr). As described above, control mice receiving a sham operation had serum Cr of 0.43±0.03 mg/dL, and those that underwent liver IR had serum Cr of 1.1±0.1 mg/dL, but Sg1P treatment almost eliminated the post-liver IR rise in Cr (0.528±0.07 mg/dL). When combined with VPC 23019 pre-treatment, Sg1P treatment failed to reduce the post-liver IR rise in serum Cr (1.4±0.4 mg/dL). Similar results were obtained when Sg1P treatment was preceded by the administration of 0.05 mg/kg W146, a more specific S1P1 antagonist (FIG. 16A). Serum Cr following liver IR mice pre-treated with W146 was 1.6±0.2 mg/dL, and Sg1P treated mice still displayed elevated serum Cr (1.5±0.2 mg/dL). In addition, VPC23019 and W146 pretreatment alone exacerbated kidney injury resulting from liver IR. See Table 5 for a summary of the results. Thus, the therapeutic action of Sg1P is mediated by the 51P1 receptor.

TABLE 5
Sg1P treatment with VPC 23019 or W146 does not protect kidney

| | Mean Creatinine | | |
|---|---|---|---|
| | (mg/dL) | S.E. | N |
| Sham + Vehicle | 0.43 | 0.03 | 6 |
| Hepatic IR + Vehicle | 1.08 | 0.07 | 6 |
| Hepatic IR + Sg1P | 0.55 | 0.05 | 6 |
| Hepatic IR + VPC | 1.61 | 0.18 | 6 |
| Hepatic IR + VPC + Sg1P | 1.42 | 0.36 | 4 |
| Hepatic IR + W146 | 1.55 | 0.18 | 7 |
| Hepatic IR + W146 + Sg1P | 1.54 | 0.17 | 7 |

In addition, we demonstrated that the therapeutic action of Sg1P administration in reducing liver injury due to liver IR is also mediated by the S1P1 receptor (see Table 6). When preceded by the administration of VPC 23019, a pharmacological antagonist of S1P1 and S1P3 receptors (50-fold more selective for S1P1), Sg1P treatment failed to reduce liver injury induced by liver IR (as measured by serum ALT). With VPC 23019 pre-treatment, serum ALT was 18253±1709 U/L 24 hours following liver IR, higher than serum ALT in control mice, i.e., subjected to liver IR receiving vehicle treatment (15076±1174 U/L), and Sg1P treatment failed to reduce the post-liver IR rise in serum ALT (19796±3418 U/L). By comparison, control mice receiving a sham operation had serum ALT of 72±9 U/L, and Sg1P treatment significantly suppressed the post-liver IR rise in ALT (7474±557 U/L). Similar results were obtained Sg1P treatment was preceded by the administration of 0.05 mg/kg (i.p.) W146, a more specific S1P1 receptor antagonist (FIG. 16B). Serum ALT following liver IR mice pre-treated with W146 was 24452±1760 U/L, and Sg1P treatment combined with W146 pre-treatment failed to suppress the liver IR-induced rise in serum ALT (22834±1411 U/L). See Table 6 for a summary of the results. In addition, VPC23019 and W146 pretreatment alone exacerbated liver injury resulting from liver IR. Pre-treatment with W146 caused dose-dependent (0.01-0.2 mg/kg) inhibition of Sg1P's protective effects against liver and kidney injury, with 0.1 mg/kg (i.p.) W146 administered 10 minutes prior to liver ischemia being sufficient to completely inhibit Sg1P-mediated renal and hepatic protection

TABLE 6
Sg1P treatment with VPC 23019 or W146 does not protect liver

| | Mean | | |
|---|---|---|---|
| | ALT (U/L) | S.E. | N |
| Sham + Vehicle | 72 | 9 | 6 |
| Hepatic IR 24 h + Vehicle | 15076 | 1174 | 6 |
| Hepatic IR 24 h + Sg1P | 7474 | 557 | 6 |
| Hepatic IR 24 h + VPC | 18253 | 1708 | 6 |
| Hepatic IR 24 h + VPC + Sg1P | 19796 | 3418 | 4 |
| Hepatic IR 24 h + W146 | 24451 | 1760 | 7 |
| Hepatic IR 24 h + W146 + Sg1P | 22834 | 1411 | 8 |

We also tested whether or not specific pharmacological antagonists to the S1P2 receptor (JTE-013; 0.1 mg/kg i.p.) or the S1P3 receptor (BML-241; 0.1 mg/kg i.p.) would also prevent sphinganine 1-phosphate-mediated renal and hepatic protection after liver IR. We found that blockade of S1P2 or S1P3 receptors did not block the Sg1P-mediated liver and kidney protection after liver IR. (FIGS. 16A and B). Thus, it appears that specific activation of the endogenous S1P1 receptor by Sg1P is protective against liver-IR-induced injury. In fact, SEW2871, a selective S1P1 receptor agonist (1 mg/kg i.p. 30 min. prior to liver ischemia and 5 min prior to reperfusion) also provided liver (ALT=6502±552 U/L, N=6) and renal (Cr=0.63±0.08 mg/dL, N=6) protection to a degree equivalent to sphinganine-1-phosphate treatment. Similar protection from liver IR-induced injury was observed in treatment with another S1P1 receptor agonist, FTY720. FTY720 treatment (1 mg/kg i.p. 30 min. prior to liver ischemia and 5 min prior to reperfusion), resulted in serum ALT of 6435±944 (N=3) and serum Cr of 0.65±0.06 mg/dL (N=3).

We conducted further studies to elucidate the cellular mechanism(s) of sphinganine 1-phosphate-mediated liver and kidney protection after liver IR. Activation of 51P1 receptors in vascular endothelial cells initiates several cytoprotective kinase signaling cascades including ERK mitogen activated protein kinase (ERK/MAPK) and Akt via pertussis toxin-sensitive G-protein (Gi/o) dependent pathway. ERK/MAPK and Akt signaling pathways are known to protect against endothelial cell apoptosis and since hepatic IR induced acute kidney injury (AKI) directly causes renal endothelial cell apoptosis with subsequent vascular dysfunction and neutrophil infiltration, we hypothesized that the sphinganine 1-phosphate via S1P1 receptor-mediated activation of ERK/MAPK and Akt signaling pathways protect against renal endothelial cell apoptosis and reduce AKI after liver IR. In addition, we have shown previously that enhanced phosphorylation as well as increased synthesis of heat shock protein 27 (HSP27) protected against endothelial cell apoptosis and vascular compromise after hepatic IR. Therefore, we postulated that Sg1P may also increase HSP27 phosphorylation and upregulation. Finally, since endothelial nitric oxide synthase (eNOS) upregulation with subsequently enhanced release of NO protects against vascular endothelial cell injury and S1P receptor activation is known to activate eNOS to increase NO levels in the vasculature, we postulated that sphinganine 1-phosphate-mediated activation of S1P1 receptors may protect against liver and kidney injury via stimulating the eNOS pathway.

Our results show that activation of the S1P1 receptors via sphinganine 1-phosphate protects against liver IR induced AKI and hepatic injury via Gi/o, ERK and Akt-mediated mechanisms and the protection is independent of the eNOS pathway. Moreover, sphinganine 1-phosphate phosphorylated and upregulated HSP27.

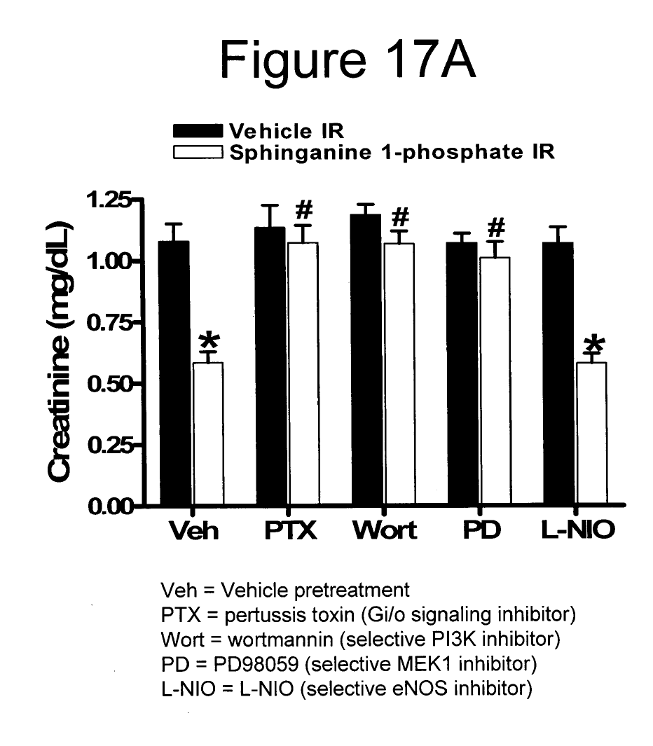

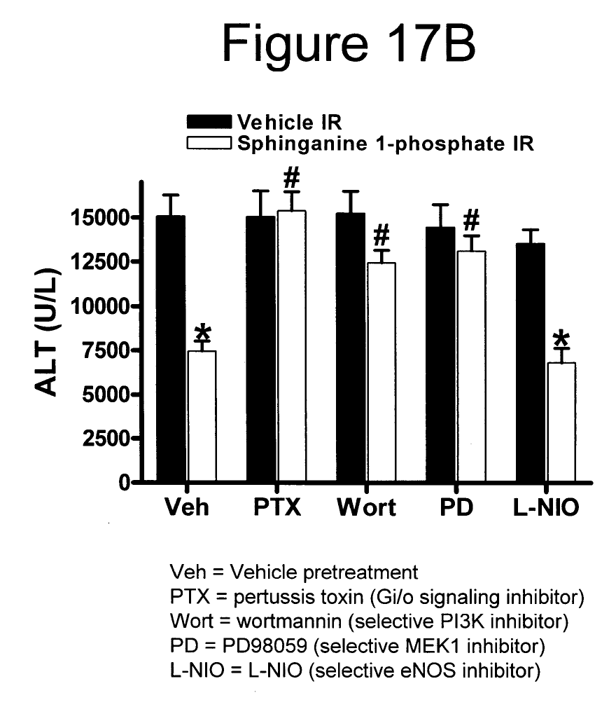

We probed the renal and hepatic protective signaling pathways activated by sphinganine 1-phosphate treatment in mice subjected to liver IR. To determine whether Gi/o, ERK/MAPK, Akt and/or eNOS signaling mediate the sphinganine 1-phosphate-mediated renal and hepatic protection after liver IR, mice were pretreated with pertussis toxin (an inhibitor of Gi/o signaling), PD98059 (a selective MEK1 inhibitor), wortmannin (a selective PI3K inhibitor) or L-NIO (a selective eNOS inhibitor) prior to sphinganine 1-phosphate treatment. We have previously determined the doses of pertussis toxin, PD98059 and wortmannin effective in blocking phosphorylation of ERK and Akt, respectively, in mice in vivo (Joo J D, et al. Am J Physiol Renal Physiol 2007 December; 293(6):F1847-F1857.; Joo J D, et al. J Am Soc Nephrol 2006 November; 17(11):3115-3123). We tested serum creatinine and ALT in these mice, and found that the inhibition of Gi/o, MEK1 or PI3K prevented the renal and hepatic protection normally provided by sphinganine 1-phosphate treatment after hepatic IR (FIGS. 17A and B). The selective eNOS inhibitor L-NIO had no effects on sphinganine 1-phosphate-mediated renal and hepatic protection after liver IR (FIGS. 17A and B). Inhibitors alone (without sphinganine 1-phosphate) had no effect on renal or hepatic function after IR injury (FIGS. 17A and B, black bars).

H&E slides of livers taken from mice subjected to liver IR were also analyzed for the degree of hepatocellular damage using the Suzuki's criteria. Pre-treating mice with W146, PD98059, wortmannin or pertussis toxin prior to sphinganine 1-phosphate treatment reduced the protective effects of sphinganine 1-phosphate on the hepatic histology of mice subjected to liver IR. See Table 7. Necrotic areas in the liver after IR also increased significantly in mice treated with W146, PD98059, wortmannin or pertussis toxin.

TABLE 7
Sg1P treatment combined with W146, PD98059, wortmannin or
pertussis toxin pretreatment is not protective

| Suzuki score | | |
|---|---|---|
| (0 = no injury, 12 = maximal injury) | Mean | N |
| Sham (C57) | 0 | 5 |
| Hepatic IR + Vehicle | 8.7 ± 0.3 | 5 |
| Hepatic IR + Sg1P | 5.2 ± 0.8 | 5 |
| Hepatic IR + Sg1P + W146 | 9.8 ± 0.6 | 5 |
| Hepatic IR + Sg1P + PD98059 | 9.3 ± 0.5 | 5 |
| Hepatic IR + Sg1P + wortmannin | 9.8 ± 0.3 | 5 |
| Hepatic IR + Sg1P + Pertussis toxin | 9.0 ± 0.6 | 5 |

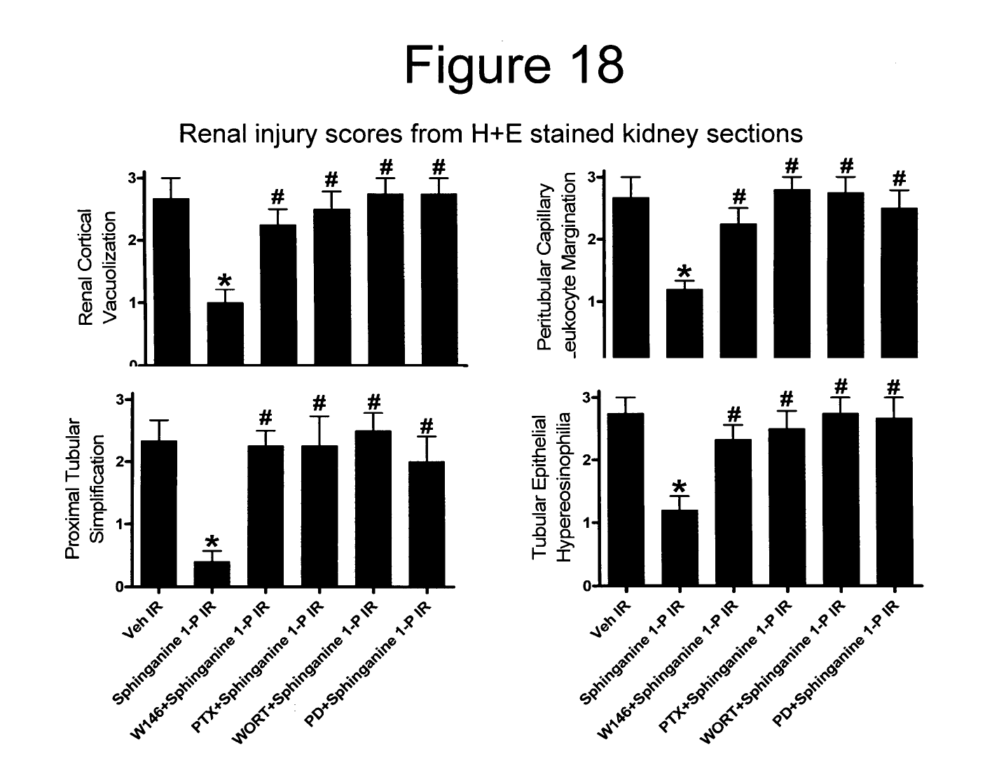

Similarly, blockade of S1P1 receptors, MEK1, PI3K or Gi/o by pre-treating mice with W146, PD98059, wortmannin or pertussis toxin, respectively, prior to sphinganine 1-phosphate treatment reduced the protective effects of sphinganine 1-phosphate on the renal histology of mice subjected to liver IR (FIG. 18).

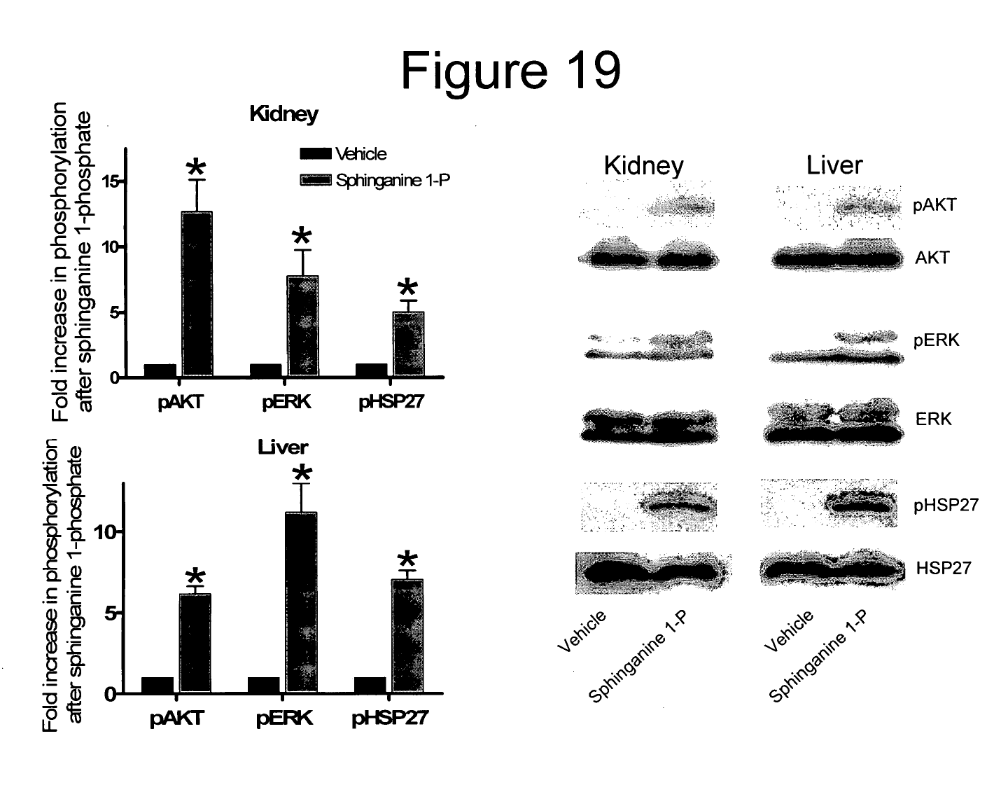

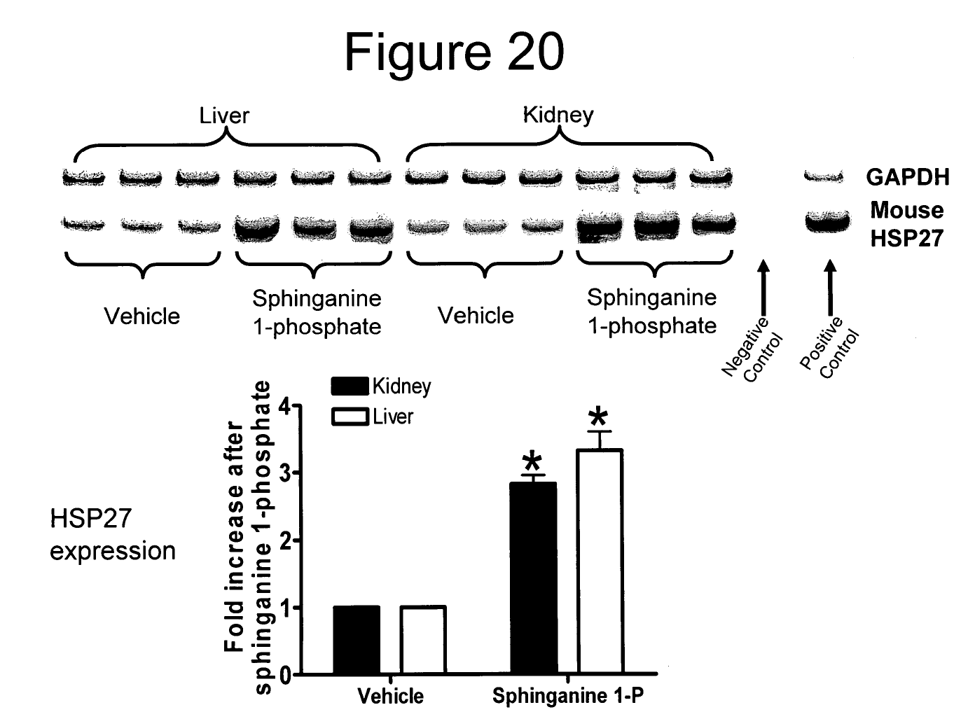

Mice were injected with sphinganine 1-phophate intravenously and their kidney and liver tissues were extracted at 15 minutes after injection (for immunoblotting of phosphorylated proteins) or at 5 hours after injection (for RT-PCR). Sphinganine 1-phosphate treatment resulted in phosphorylation of ERK/MAPK, Akt and phosphorylation of HSP27 in the liver and kidney of these mice (FIG. 19). Sphinganine 1-phosphate also induced HSP27 mRNA expression in the liver and kidney (FIG. 20). HSP27 is a known F-actin stabilizing and anti-apoptotic protein.

### Example 6

### Renal Damage after Liver IR is Worse in Mice Lacking Sphingosine Kinase (SK), the Enzyme Responsible for Sg1P Synthesis

We demonstrated that endogenous plasma level of Sg1P falls after liver IR and that exogenous Sg1P protects against liver-IR-induced AKI in mice. This may be due to a decrease in renal and systemic ATP levels after liver IR (Zager, 1991) and the subsequent inability to maintain normal function of sphingosine kinase (SK) enzyme, the synthetic enzyme for Sg1P.

To further demonstrate a cytoprotective role for Sg1P against liver IR induced AKI, and to determine the SK subtype responsible for generating cytoprotective Sg1P, mice genetically deficient (KO) for SK were studied (Mizugishi et al., 2005; Allende et al., 2004).

Utilizing HPLC analysis, plasma levels of Sg1P in KO mice deficient for SK1 or SK2 were measured to determine whether a deficiency in either of these SK would lead to reduced plasma levels of Sg1P. As described above in Table 1, plasma levels of Sg1P decreased significantly in mice subjected to liver IR compared to the sham-operated mice. A deficiency of either SK1 or SK2 also led to reduced plasma levels of Sg1P, as shown in Table 8.

Control SK1 KO mice with sham surgeries had lower Sg1P levels in comparison (0.16±0.04 fold WT sham, N=6), as did control SK2 KO mice (0.56±0.54 fold WT sham, N=6) compared to C57BL/6 wildtype. Measurement of Sg1P levels 24 hours post-liver IR showed liver IR resulted in increased (but still low) Sg1P levels for both SK1 KO mice (0.37±0.04 fold WT sham, N=7) and SK2 KO mice (0.68±0.04 pmol/ul plasma, N=9, as opposed to the reduction in serum Sg1P seen in wildtype mice. See Table 8 for a summary of results.

TABLE 8
Serum Sg1P levels in Sphingosine Kinase-deficient mice

| | SK1 KO | | SK2 KO | | WT (C57BL/6) | |
|---|---|---|---|---|---|---|
| | Sham | Liver IR | Sham | Liver IR | Sham | Liver IR |
| Serum Sg1P | 0.16 | 0.37 | 0.56 | 0.68 | 1.00 | 0.44 |
| (fold WT | | | | | | |
| sham) | | | | | | |
| SE | 0.04 | 0.04 | 0.54 | 0.04 | 0.05 | 0.11 |
| N | 6 | 7 | 6 | 9 | 21 | 8 |

To determine whether SK1 and SK2 KO mice subjected to liver IR injury demonstrated enhanced liver and renal injury, plasma levels of ALT and creatinine, respectively, were measured. The results are shown in Tables 9 and 10 below. The effects of administering Sg1P (0.1 mg/kg i.v. and 0.2 mg/kg s.c.) on liver and renal injury were also determined in the KO mice and are also shown in Tables 9 and 10 below.

SK1 and SK2-deficient mice subjected to liver IR showed enhanced liver injury as evidenced by the higher plasma levels of ALT compared to C57BL/6 wildtype mice subjected to liver IR (compare Table 9 with Table 6). Sg1P administration reduced liver IR-induced liver damage in mice deficient in SK1, as well as SK2-deficient mice. S1P administration somewhat reduced liver IR-induced liver damage in mice deficient in SK2-deficient mice, but failed to have any protective effect on the liver in SK1-deficient mice (see Table 9). Sphinganine treatment failed to provide protective effect on the liver in SK-deficient mice (data not shown).

TABLE 9
Liver IR-induced hepatic injury in SK-deficient mice

| | | | SK1KO | SK1KO | | SK2KO | SK2KO |
|---|---|---|---|---|---|---|---|
| | | SK1KO | hepatic | hepatic | SK2KO | hepatic | hepatic |
| ALT | sham | hepatic | IR 24 h + | IR 24 h + | hepatic | IR 24 h + | IR 24 h + |
| (U/L) | (SK2KO) | IR 24 h | Sg1P | S1P | IR 24 h | Sg1P | S1P |
| Mean | 48.42 | 17891.84 | 9602.01 | 17416.67 | 20207.41 | 7575.28 | 13165.08 |
| S.E. | 9.29 | 977.20 | 3923.81 | 5105.94 | 1860.66 | 2091.40 | 1424.92 |
| N | 3 | 7 | 3 | 3 | 9 | 7 | 3 |
| P vs Sham | n/a | n/a | n/a | n/a | 0.0001 | 0.0524 | 0.0008 |
| P vs. IR | | n/a | 0.0184 | 0.8929 | n/a | 0.0005 | 0.0657 |

SK1 and SK2-deficient mice subjected to liver IR also showed enhanced renal injury as evidenced by the higher plasma levels of creatinine compared to C57 wildtype mice subjected to liver IR (compare Table 10 with Table 5). Sg1P administration reduced liver IR-induced renal damage in mice deficient in SK1, as well as SK2-deficient mice (see Table 10). S1P also provided renal protection, but at weaker levels compared to Sg1P. Sphinganine treatment failed to provide kidney protection in SK-deficient mice (data not shown).

TABLE 10
Liver IR-induced renal injury in SK-deficient mice

| | | SK1KO | SK1KO | | | |
|---|---|---|---|---|---|---|
| | | hepatic | hepatic | | SK2KO | SK2KO |
| | SK1KO | IR 24 | IR 24 | SK2KO | hepatic | hepatic |
| Creatinine | hepatic | h + | h + | hepatic | IR 24 h + | IR 24 h + |
| (mg/dL) | IR 24 h | Sg1P | S1P | IR 24 h | Sg1P | S1P |
| Mean | 1.706 | 0.709 | 1.035 | 1.548 | 0.679 | 0.880 |
| SE | 0.159 | 0.396 | 0.330 | 0.234 | 0.103 | 0.194 |
| N | 3 | 3 | 3 | 9 | 7 | 3 |
| P vs. IR | n/a | 0.0795 | 0.1407 | n/a | 0.0082 | 0.1519 |

### Example 7

### Over-Expression of SK Using Lentiviral Vector Treats Renal and Liver Failure Due to Liver IR in Mice

To determine whether renal over-expression of the enzyme that synthesizes Sg1P would protect against AKI after liver IR, an in vivo lentiviral-mediated renal expression method (as described by Nakamura et al. (2004)) was used to overexpress SK1 or SK2 in the kidney of mice subjected to liver IR. Lentiviral vectors encoding SK1 or SK2 were generated by subcloning SK1 or SK2 plasmid (provided by Dr. Stuart Pitson, Hanson Institute, Australia) into a shuttle vector (pLL3.7) and transfecting HEK293-FT cells with Vesicular stomatitis virus G (Invitrogen) and pA8.9 (from Dr. Jay Yang, Anesthesiology). Vesicular stomatitis virus G (VSVG)-pseudotyped human immunodeficiency virus (HIV) vectors were generated by cotransfecting the lentivirus shuttle vector (Δu6-pLL-IRES-EGFP-SK1 or SK2) with the HIV-1 packaging vector pCMVΔR8.91 and the pMD.G plasmid encoding the VSVG envelope glycoprotein into HEK-293FT cells. Briefly, 10 μg of Δu6-pLL-IRES-EGFP-SK1 or SK2, 5 μg of pCMVΔR8.91, and 7 μg of pMD.G were cotransfected into 80-90% confluent HEK-293FT cells in 10-cm tissue culture plates using 20 μl of Lipofectamine 2000 (Invitrogen, Carlsbad, Calif.) in serum-free OptiMEM medium according to the manufacturer's recommendations. After 6 h, the medium was replaced with DMEM supplemented with 10% FBS and penicillin/streptomycin (Pen-Strep). The cell culture medium from the HEK-293FT cells was collected after 72 h and passed through a 0.45-μm filter, and the virus titer was increased ˜10-fold by ultrafiltration (Amicon Ultra 100,000 MW cutoff; Millipore, Bedford, Mass.). The lentivirus preparation was injected into the kidney two days prior to liver IR. In anesthetized mice, a 31G needle was inserted at the lower pole of the kidney perpendicular to the long axis and carefully pushed toward the upper pole. As the needle was slowly removed, 100 μl virus was injected.

C57BL/6 WT mice that received intrarenal injection of SK-encoding lentiviral vector showed elevated serum Sg1P. Mice treated with SK1-encoding lentiviral vector had serum Sg1P levels that was almost twice that of controls (1.91±0.33 fold sham; N=3; p=0.0001). To determine whether intrarenal injection of SK1-encoding lentiviral vector reduced renal and liver injury in liver IR mice, plasma levels of creatinine and ALT, respectively, were measured and are shown in Tables 10 and 11 below.

Intrarenal injection of SK1- or SK-2 encoding lentiviral vector prevented the liver-IR-induced increase in serum creatinine. While liver IR in control mice treated with intrarenal administration of EGFP-encoding lentiviral vector resulted in serum creatinine increasing from 0.543±0.033 mg/dL (control) to 1.055±0.119 mg/dL, mice who received an intrarenal injection of SK1- or SK2-encoding lentiviral vector showed no significant rise in serum creatinine when subjected to liver IR (i.e., the mouse was protected from renal injury) (see Table 10). The results in Table 10 demonstrate that intrarenal administration of lentiviral vector encoding SK1 or SK2 reduces and treats renal failure due to liver IR. Intraperitoneal injection of the SK1-encoding lentiviral vector did not reduce the liver-IR-induced rise in serum creatinine, demonstrating that intrarenal injection is a preferred method of administering the therapeutic virus.

TABLE 10
Intrarenal administration of lentiviral vector encoding SK protects renal
function in mice subjected to liver IR
mg/dL Serum creatinine levels in C57BL/6 mice

| | sham | | | | | |
|---|---|---|---|---|---|---|
| | | | SK1- | Liver IR | | |
| | EGFP- | EGFP- | lentivirus | SK1- | SK2- | Sg1P |
| | lentivirus | lentivirus | intra- | lentivirus | lentivirus | treat- |
| | intrarenal | intrarenal | peritoneal | intrarenal | intrarenal | ment |
| Mean | 0.543 | 1.055 | 1.316 | 0.588 | 0.544 | 0.55 |
| SE | 0.033 | 0.119 | 0.194 | 0.101 | 0.036 | 0.05 |
| N | 4 | 13 | 4 | 7 | 6 | 6 |

Similarly, intrarenal injection of SK1- or SK2-encoding lentiviral vector inhibited the liver-IR-induced increase in serum ALT (see Table 11). While liver IR in control mice treated with intrarenal administration of EGFP-encoding lentiviral vector resulted in serum ALT increasing from 47±18 U/L to 15432±752 U/L, mice that received an intrarenal injection of SK1-encoding lentiviral vector had their serum ALT only increase to 5046±1664 U/L when subjected to liver IR (i.e., the mouse was protected from liver injury). Similar results were obtained with SK2-encoding lentiviral vector. See Table 11. The results in Table 11 demonstrate that intrarenal administration of virus encoding SK1 or SK2 reduces and treats liver failure resulting from liver IR. In fact, the SK-encoding lentiviral vector treatments were more effective in protecting the liver from IR-related injury than direct (i.p. and s.c.) Sg1P administration. Intraperitoneal injection of the SK1-encoding lentiviral vector did not reduce the liver-IR-induced rise in serum ALT, demonstrating that intrarenal injection is a preferred method of administering the therapeutic virus.

TABLE 11
Intrarenal administration of lentiviral vector encoding SK protects
hepatic function in mice subjected to liver IR
U/L Serum ALT levels in C57BL/6 mice

| | sham | | | | | |
|---|---|---|---|---|---|---|
| | | | SK1- | liver IR | | |
| | EGFP- | EGFP- | lentivirus | SK1- | SK2- | Sg1P |
| | lentivirus | lentivirus | intra- | lentivirus | lentivirus | treat- |
| | intrarenal | intrarenal | peritoneal | intrarenal | intrarenal | ment |
| Mean | 47.01 | 15432 | 25762 | 5046 | 4098 | 7474 |
| SE | 17.68 | 752 | 2525 | 1664 | 608 | 557 |
| N | 4 | 17 | 4 | 7 | 6 | 6 |

The results demonstrate that renal overproduction of Sg1P, via overexpression of SK1 or SK2, rescued renal function and liver function in mice following liver IR.

### Example 8

### Addition of Sg1P to Cultured Human Cells Inhibits Cell Death

We conducted further experiments to determine that Sg1P treatment had the same protective effects in human cells as in mice. Since free radicals as well as increased TNF-α are implicated in initiating multiorgan dysfunction after liver IR (Tsung et al., 2005) and the preliminary data confirmed that plasma TNF-α significantly increased after liver IR and that H₂O₂ caused significant apoptosis in human renal endothelial cells, exogenous Sg1P was tested for its ability to protect against endothelial cell damage (apoptosis, F-actin degradation as well as increased transcellular permeability) after TNF-α or H₂O₂ treatment.

IHUVEC—immortalized human umbilical vein endothelial cells (ATCC, P.O. Box 1549 Manassas, Va. 20108) or HRGEC—human renal glomerular endothelial cells (ScienCell Research Laboratories, 6076 Corte Del Cedro Carlsbad, Calif. 92011) were cultured within a humidified atmosphere of 5% CO2 in air and grown to confluence. When confluent, the cells were trypsinized and seeded onto 0.45 μm pore size polycarbonate filters at the base of inserts of 6.5 mm diameter Transwell chambers and cultured until confluence.

Human endothelial cell permeability was assessed with the Evans blue dye extravasation assay (Awad et al., 2006). The permeability characteristics of the resulting confluent monolayers were assessed by following the diffusion of albumin-conjugated Evan's blue dye from the upper to lower compartments of the Transwell chambers. After addition of dye, chambers were shaken and allowed to equilibrate for 10-30 min at 37° C. After equilibration, stimuli were added and dye levels in lower compartments intermittently assayed by spectrophotometric analysis.

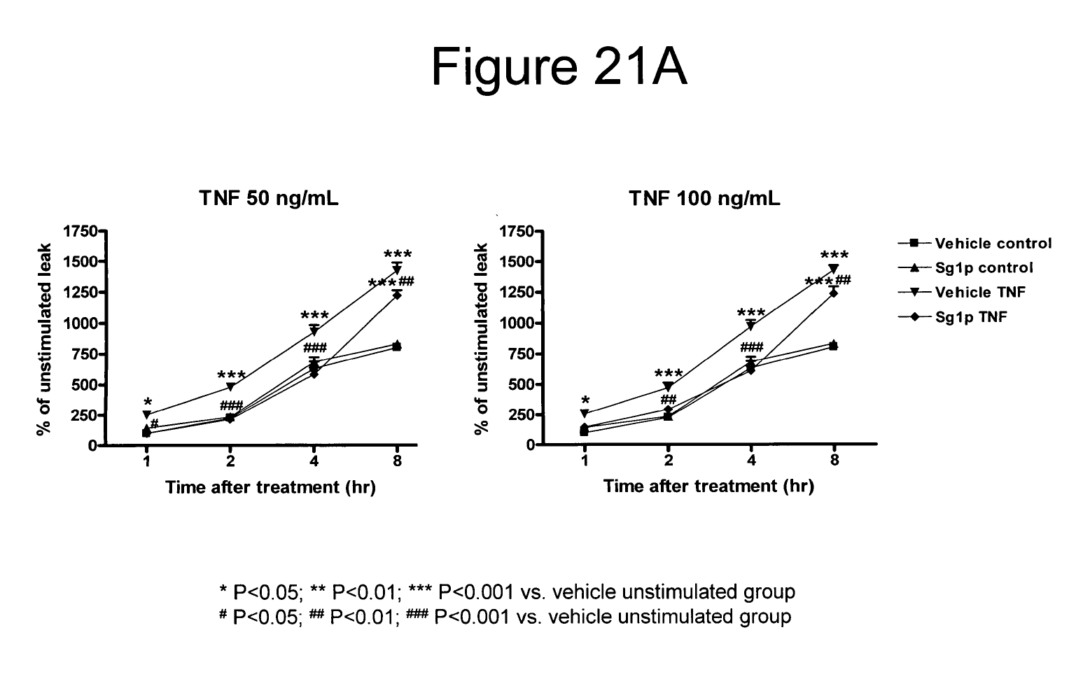

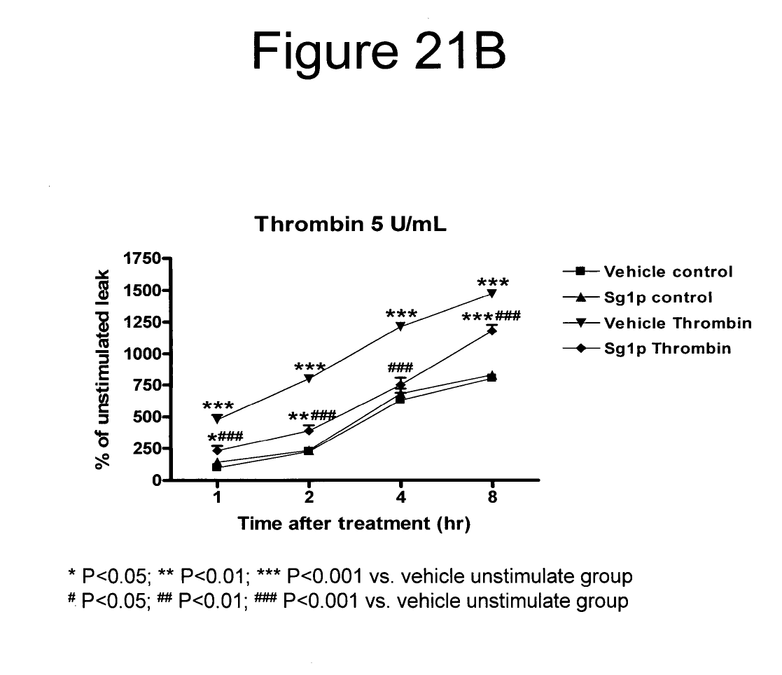

The addition of TNF-α or thrombin induces apoptosis and increases cell permeability in cultured endothelial cells. IHUVEC cells that were treated with TNF-α (50 ng/ml or 100 ng/ml) displayed increased permeability compared to controls, as measured by the Evans blue dye extravasation assay, as shown in FIG. 21A. However, concurrently treating the cultures with Sg1P reduced the permeability of the cells to levels comparable to control (not treated with TNF-α). The same cytoprotective effect of Sg1P was seen when apoptosis was induced with 5 U/ml of thrombin, as shown in FIG. 21B.

In further experiments, apoptosis was induced in human renal endothelial (HRGEC) cells with either TNF-α plus cycloheximide or H₂O₂, with or without Sg1P treatment. In both cases, the addition of Sg1P (1 uM) reduced PARP and caspase 3 fragmentations, which are indices of apoptosis (N=2, data not shown).

### Example 9

### Sg1P Protects Against Human Endothelial Cell Apoptosis Via Stabilization of F-Actin Through ERK and Akt Phosphorylation and HSP27 Induction

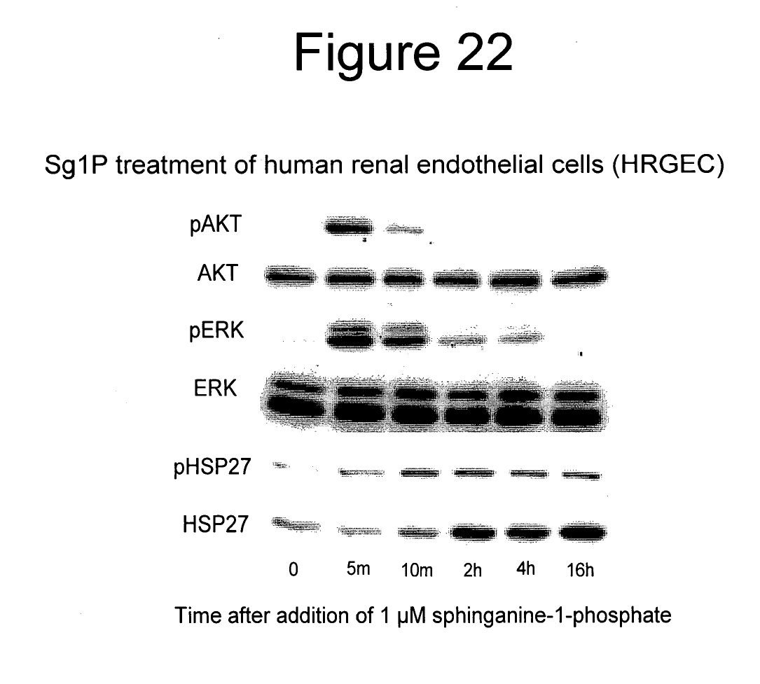

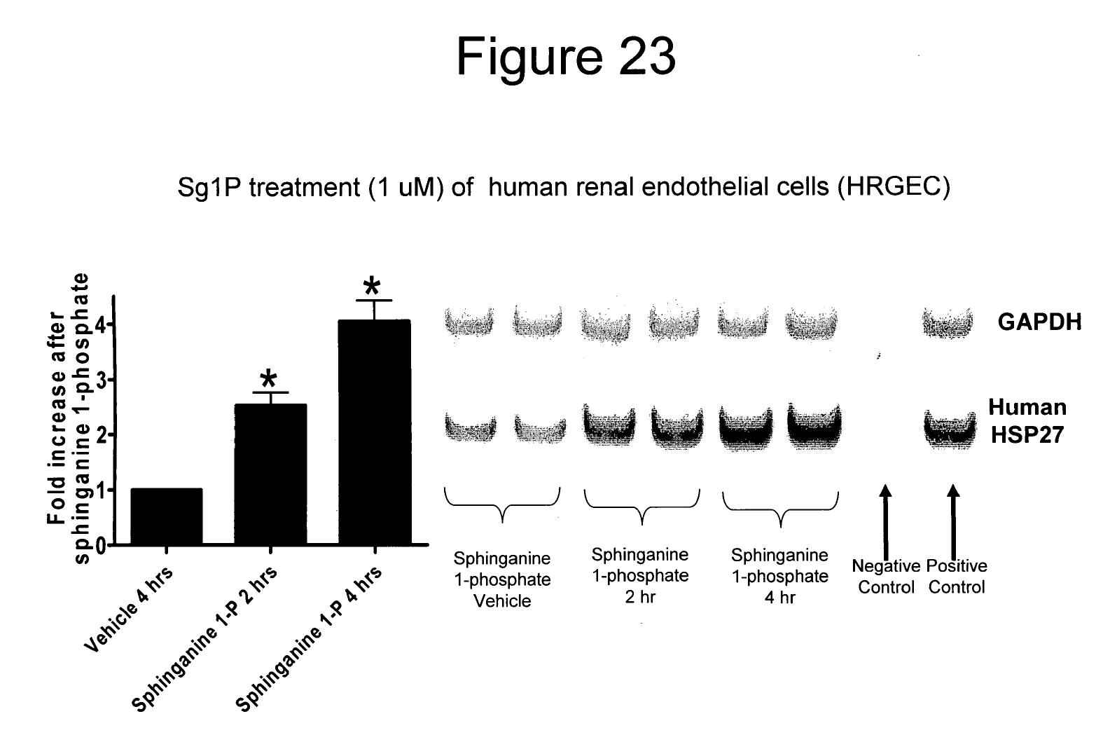

As shown in FIG. 22, Sg1P treatment in HRGEC also resulted in the phosphorylation of two well known anti-apoptotic kinases (ERK and Akt). Moreover, Sg1P induced the phosphorylation of HSP27, as well as increased expression of total HSP27, a known F-actin stabilizing and anti-apoptotic molecule (FIG. 22; FIG. 23). Thus, we demonstrated that exogenous Sg1P protects against human endothelial cell dysfunction by phosphorylating anti-apoptotic ERK, Akt and HSP27, as well as increasing HSP27 expression.

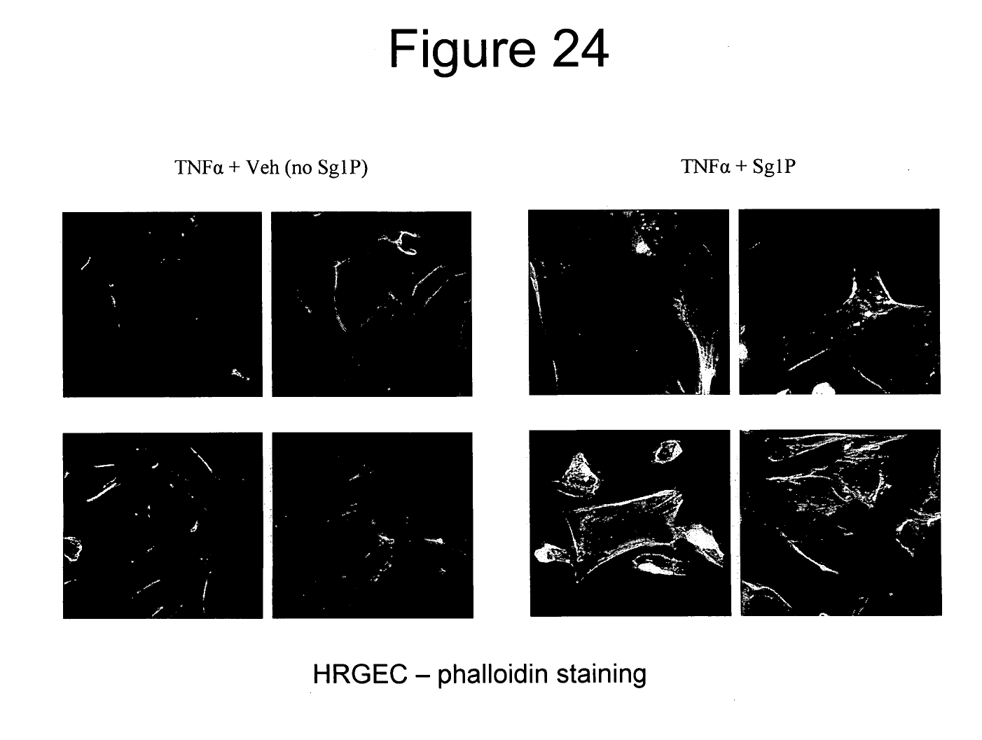

To further demonstrate the anti-apoptotic property of Sg1P, F-actin stability in HRGEC cells was examined with fluorescent phalloidin staining. The addition of TNF-α resulted in reduced phalloidin staining in HRGEC cells (N=4). However, co-administration of Sg1P restored the phalloidin staining (N=4), indicating F-actin stabilization and protection from apoptosis. See FIG. 24.

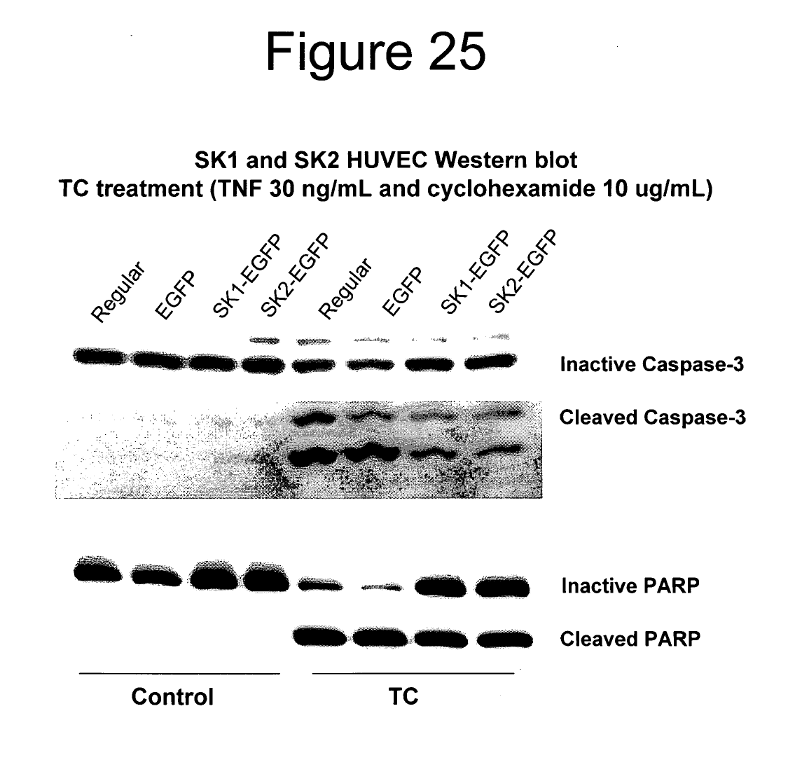

Overexpression of SK (SK1 or SK2) was examined to determine if it protects against human endothelial cell death in vitro. Human umbilical vein endothelial cells (HUVEC) were infected with lentiviral vector encoding SK1 or SK2. Cells stably overexpressing SK were sorted by fluorescence and subjected to TNF-α (30 ng/mL) with cyclohexamide (10 μg/mL). Apoptosis, as indicated by caspase 3 fragmentation, was attenuated in endothelial cells overexpressing SK, compared to controls (untreated or treated with EGFP-encoding lentiviral vector). See FIG. 25. SK overexpression also reduces TNF-α-induced transcellular permeability and F-actin degradation. Similar protective effects are seen with HSP27 overexpression.

As shown above, Sg1P protects human endothelial cells, and does so through the same mechanism as shown in mouse tissue. In contrast to the effects on human endothelial cells, sphinganine 1-phosphate failed to phosphorylate ERK/MAPK, Akt and HSP27 and also failed to induce HSP27 expression in the human epithelial HK-2 cell line (data not shown).

### Example 10

### Improved Kidney Function with Sg1P Treatment Helps Reduce Liver Injury Following Liver IR

Since both renal and liver injury are reduced with Sg1P treatment, it was a question whether Sg1P treatment healed the liver, which then helped the kidneys, or the other way around. We determined that lack of proper kidney function increased the damage liver IR inflicted on the liver.

Normally, 1 hour of liver IR results in serum ALT increasing to 15076±1174 U/L (N=6) 24 hours later. In nephrectomized mice, however, the rise in ALT was more severe, and it rose to 20237±2628 U/L (N=3) after only 45 minutes of liver IR. See Table 12 below. The nephrectomized mice were also less responsive to Sg1P treatment. In control mice without nephrectomy, Sg1P treatment reduced the rise in serum ALT levels to 7474±557 U/L, compared to ALT levels of 15076±1174 U/L in the group without Sg1P treatment. Thus, in non-nephrectomized mice, the change in ALT levels shows a 50% reduction in the severity of liver injury with Sg1P treatment. By contrast, untreated nephrectomized mice had serum ALT levels of 20236±2628 U/L 24 hours after liver IR, whereas those treated with Sg1P had serum ALT levels of 17029±1124 U/L. Thus, in nephrectomized mice, the change in ALT levels shows a 16% reduction in the severity of liver injury with Sg1P treatment. See Table 12 below.

Therefore, the nephrectomized mice showed attenuated protection against liver IR injury with Sg1P and suggesting an important role for the kidney in mediating protection of both the liver and the kidney with Sg1P.

TABLE 12
Nephrectomized C57BL/6 mice with 45 min. liver IR

| | IR | IR + Sg1P |
|---|---|---|
| ALT U/L | 22925.4 | 15993.65 |
| (24 hrs. post-IR) | 22803.17 | 19276.19 |
| | 14980.95 | 15819.05 |
| Mean | 20236.51 | 17029.63 |
| SE | 2628.015 | 1124.411 |

### Example 11

### Serum Sg1P Levels Drop after Liver Transplantation in Human Subject

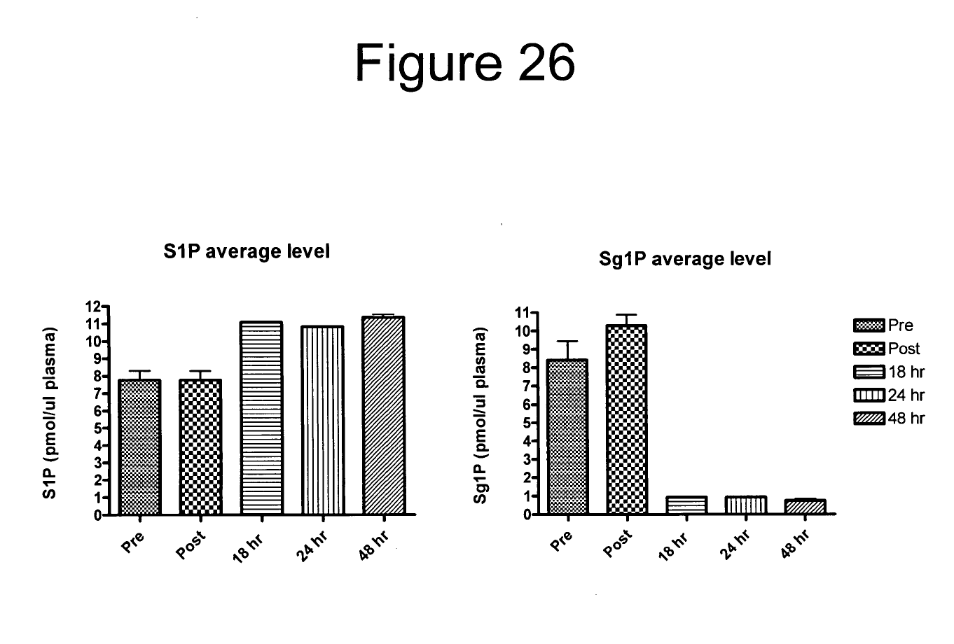

Renal failure is a common postoperative complication following non-renal organ transplantation, including, but not limited to, liver transplant surgery (Ojo et al., 2003, Doddakula et al., 2007). To test if liver transplantation results in the lowering of Sg1P levels, and whether or not low Sg1P levels correlate to poor post-transplantation outcome, Sg1P levels in the blood were monitored at regular intervals in liver transplant recipients before and after the transplantation surgery. As shown in FIG. 26, we found that serum Sg1P average levels dropped in patients following liver transplantation and remained at a low level until at least 48 hours post transplantation In contrast, serum S1P average levels did not drop in patients following liver transplantation (see FIG. 26). These results demonstrate that Sg1P, but not S1P, is reduced in human patients following liver IR.

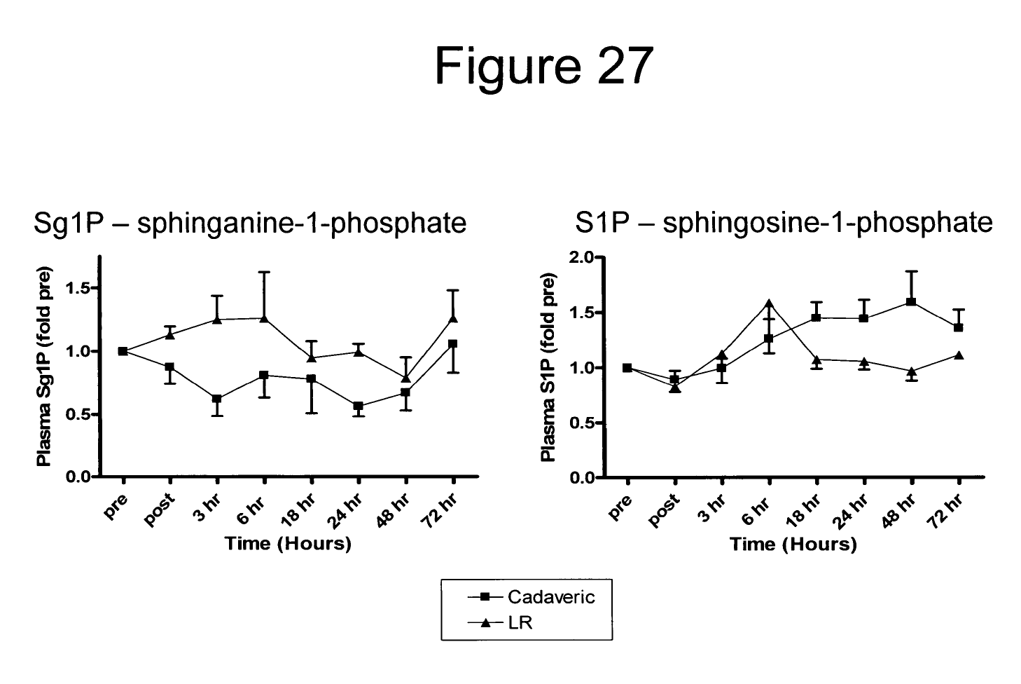

It was further observed that the drop in Sg1P levels was more significant when patients received livers from cadaveric donors rather than liver from live donors. Blood Sg1P levels were stable in patients that received a live-donor liver (N=7), whereas in patients receiving cadaveric liver, Sg1P levels decreased to approximately 60% of pre-operation levels within 6 hours, and did not recover to pre-operation levels until 72 hours post-operation (N=24; see FIG. 27). By contrast, S1P levels measured from the same samples did not fall below pre-operation levels. In fact, serum S1P levels rose approximately 50% in patients receiving cadaveric livers (see FIG. 27). The success of liver transplantation is higher when the transplanted liver is from a live donor, as opposed to a cadaveric donor. One contributing factor in the difference in outcome is that cadaveric livers experience a higher degree of ischemia reperfusion injury compared to live-donor livers. Our results demonstrate that patients receiving a cadaveric liver has a stronger reduction in the recipients' Sg1P levels, and is therefore correlated with poorer post-operative outcome. Thus, Sg1P levels may be used as a diagnostic for monitoring the renal health of liver transplantation patients, and as a predictor of transplantation success and patient outcome/mortality. This is superior to current methods (e.g., monitoring serum creatinine), since Sg1P is an earlier marker, and its serum level changes when renal damage is less extensive.

### Example 12

### Reduction in Blood Sg1P Following Coronary Bypass Surgery is Linked to Worse Post-Operative Outcome

Renal failure is a common postoperative complication following non-renal organ transplant, and is not limited to liver transplant surgery (Ojo et al., 2003, Doddakula et al., 2007). In addition, renal failure is a common problem following cardiac surgery (Kishore et al., 2007).

Blood Sg1P levels in patients who underwent cardiac bypass surgery were measured to determine if postoperative outcome was correlated to Sg1P levels. We measured Sg1P levels from two patients, CT2 and CT3, starting from before the surgery, immediately after the surgery, three hours post surgery, then at six hour intervals until two days post-surgery.

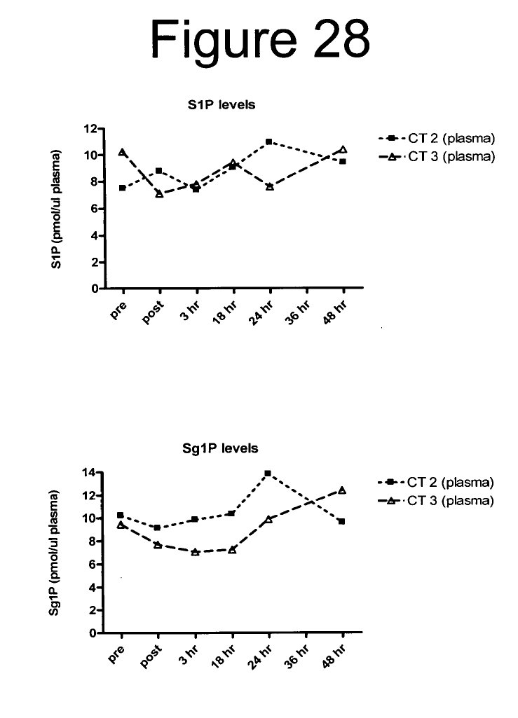

Patient CT2 was healthier, while patient CT3 was less healthy. This difference was reflected in their respective blood Sg1P levels, as shown in FIG. 28. Sg1P levels in the healthier CT3 remained higher that in the less healthy CT2. For example the pre-operation Sg1P level of CT3 was comparable (10.2 pmol/μL plasma) to that of CT2 (9.7 pmol/μL plasma). However, 18 hours after the surgery, CT3's Sg1P level decreased to 7.0 pmol/μL plasma while in the healthier CT2, it stayed steady. By contrast, the level of SP1 in the same patients remained relatively the same throughout pre- and post-operative periods. Therefore, Sg1P levels in the patients were correlated with the postoperative outcome following cardiac bypass surgery.

### REFERENCES

Allende et al. (2004), “Mice deficient in sphingosine kinase 1 are rendered lymphopenic by FTY720”, J Biol Chem. 279(50):52487-52492.
Awad et al. (2006), “Selective sphingosine 1-phosphate 1 receptor activation reduces ischemia-reperfusion injury in mouse kidney”, Am J Physiol 290:F1516-F1524.
Doddakula et al. (2007), “Predictors of acute renal failure requiring renal replacement therapy post cardiac surgery in patients with preoperatively normal renal function”, Interact CardioVasc Thorac Surg 6:314-318.
Genescà et al. (2006), “Actin cytoskeleton derangement induces apoptosis in renal ischemia/reperfusion”, Apoptosis 11:563-571.
Lee et al. (2009) “Acute kidney injury after hepatic ischemia and reperfusion injury in mice”, Laboratory Investigations, in press.
Min et al. (2002), “Simultaneous quantitative analysis of sphingoid base 1-phosphates in biological samples by o-phthalaldehyde precolumn derivatization after dephosphorylation with alkaline phosphatase”, Anal Biochem 303:167-175.
Mizugishi et al. (2005) “Essential role for sphingosine kinases in neural and vascular development”, Mol Cell Biol. 24:11113-11121.
Nakamura et al. (2004), “β2-Adrenoceptor Activation Attenuates Endotoxin-Induced Acute Renal Failure”, J Am Soc Nephrol 15:316-325.
Ojo et al. (2003) “Chronic renal failure after transplantation of a nonrenal organ”, N Engl J Med 349:931-940.
Tsung et al. (2005), “Hepatic ischemia/reperfusion injury involves functional TLR4 signaling in nonparenchymal cells”, J Immunol 175:7661-7668.
Zager (1991), “Adenine nucleotide changes in kidney, liver, and small intestine during different forms of ischemic injury”, Circ Res. 68(1):185-96.

1. A method for treating, inhibiting or preventing renal failure due to ischemia reperfusion injury in a mammalian subject in need thereof, said method comprising the step of administering to said subject a therapeutically effective amount of a therapeutic agent selected from the group consisting of sphinganine-1-phosphate, sphinganine, and a S1P1 receptor agonist.

2. The method of claim 1, wherein said ischemia reperfusion injury is associated with organ transplant, liver ischemia, kidney ischemia, stroke, abdominal aortic occlusion, abdominal aortic bleeding or perioperative ischemia due to cardiopulmonary bypass surgery.

3. (canceled)

4. (canceled)

5. (canceled)

6. The method of claim 1, wherein said S1P1 receptor agonist is selected from the group consisting of SEW2871 and FTY720.

7. (canceled)

8. (canceled)

9. The method of claim 1, wherein said mammalian subject is a human subject.

10. A method for treating, inhibiting or preventing renal failure due to ischemia reperfusion injury in a mammalian subject in need thereof, said method comprising the step of increasing sphinganine-1-phosphate levels in said subject.

11. The method of claim 10, wherein said ischemia reperfusion injury is associated with organ transplant, liver ischemia, kidney ischemia, stroke, abdominal aortic occlusion, abdominal aortic bleeding, or perioperative ischemia due to cardiopulmonary bypass surgery.

12. (canceled)

13. The method of claim 10, wherein the step of increasing sphinganine-1-phosphate levels comprises administering to said subject a sufficient amount of an agent selected from the group consisting of sphinganine-1-phosphate, sphinganine, sphingosine-1-phosphate, and sphingosine.

14. (canceled)

15. (canceled)

16. The method of claim 10, wherein the step of increasing sphinganine-1-phosphate levels comprises administering to said subject a nucleic acid encoding sphingosine kinase protein, and allowing said sphingosine kinase protein to be expressed from said nucleic acid in an amount sufficient to increase sphinganine-1-phosphate levels.

17. (canceled)

18. (canceled)

19. (canceled)

20. (canceled)

21. (canceled)

22. (canceled)

23. The method of claim 10, wherein said mammalian subject is a human subject.

24. A method for treating, inhibiting or preventing hepatic failure due to ischemia reperfusion injury in a mammalian subject in need thereof, said method comprising the step of administering to said subject a therapeutically effective amount of a therapeutic agent selected from the group consisting of sphinganine-1-phosphate, sphinganine, and a S1P1 receptor agonist.

25. The method of claim 24, wherein said ischemia reperfusion injury is associated with organ transplant, liver ischemia, kidney ischemia, stroke, abdominal aortic occlusion, abdominal aortic bleeding or perioperative ischemia due to cardiopulmonary bypass surgery.

26. (canceled)

27. (canceled)

28. (canceled)

29. The method of claim 24, wherein said S1P1 receptor agonist is selected from a group consisting of SEW2871 and FTY720.

30. (canceled)

31. (canceled)

32. The method of claim 24, wherein said mammalian subject is a human subject.

33. A method for treating, inhibiting or preventing hepatic failure due to ischemia reperfusion injury in a mammalian subject in need thereof, said method comprising the step of increasing sphinganine-1-phosphate levels in said subject.

34. The method of claim 33, wherein said ischemia reperfusion injury is associated with organ transplant, liver ischemia, kidney ischemia, stroke, abdominal aortic occlusion, abdominal aortic bleeding, or perioperative ischemia due to cardiopulmonary bypass surgery.

35. (canceled)

36. The method of claim 33, wherein the step of increasing sphinganine-1-phosphate levels comprises administering to said subject a sufficient amount of a therapeutic agent selected from a group consisting of sphinganine-1-phosphate, sphinganine, sphingosine-1-phosphate and sphingosine.

37. (canceled)

38. (canceled)

39. The method of claim 33, wherein the step of increasing sphinganine-1-phosphate levels comprises administering to said subject a nucleic acid encoding sphingosine kinase protein, and allowing said sphingosine kinase protein to be expressed from said nucleic acid in an amount sufficient to increase sphinganine-1-phosphate levels.

40. (canceled)

41. (canceled)

42. (canceled)

43. (canceled)

44. (canceled)

45. (canceled)

46. The method of claim 33, wherein said mammalian subject is a human subject.

47. A method of identifying a mammalian subject developing renal failure due to an ischemia reperfusion injury, the method comprising the steps of:
a) determining the concentration of sphinganine-1-phosphate in a biological sample from said subject who has suffered an ischemia reperfusion injury; and
b) comparing said concentration of sphinganine-1-phosphate in said subject with a reference concentration of sphinganine-1-phosphate, wherein reduced sphinganine-1-phosphate concentration in said subject compared to said reference concentration is indicative that said subject is developing renal failure.

48. (canceled)

49. (canceled)

50. (canceled)

51. (canceled)

52. (canceled)

53. (canceled)

54. A method for reducing, inhibiting or preventing kidney endothelial cell injury, said method comprising administering an effective amount of sphinganine-1-phosphate to said endothelial cells.

55. (canceled)

56. (canceled)

57. (canceled)
